SEQUENCE LISTING (52 sequences)

SEQ ID NO: 1 - protein, 805 aa, Homo sapiens [sequence omitted]

SEQ ID NO: 2 - protein, 805 aa, Mus musculus
MSSSSWLLLSLVAVTTAQSLTEENAKTFLNNFNQEAEDLSYQSSLASWNYNTNITEENAQKMSEAAAKWSAFYEEQSKTAQSFSLQEIQTPIIKRQLQALQQSGSSALSADKNKQLNTILNTMSTIYSTGKVCNPKNPQECLLLEPGLDEIMATSTDYNSRLWAWEGWRAEVGKQLRPLYEEYVVLKNEMARANNYNDYGDYWRGDYEAEGADGYNYNRNQLIEDVERTFAEIKPLYEHLHAYVRRKLMDTYPSYISPTGCLPAHLLGDMWGRFWTNLYPLTVPFAQKPNIDVTDAMMNQGWDAERIFQEAEKFFVSVGLPHMTQGFWANSMLTEPADGRKVVCHPTAWDLGHGDFRIKMCTKVTMDNFLTAHHEMGHIQYDMAYARQPFLLRNGANEGFHEAVGEIMSLSAATPKHLKSIGLLPSDFQEDSETEINFLLKQALTIVGTLPFTYMLEKWRWMVFRGEIPKEQWMKKWWEMKREIVGVVEPLPHDETYCDPASLFHVSNDYSFIRYYTRTIYQFQFQEALCQAAKYNGSLHKCDISNSTEAGQKLLKMLSLGNSEPWTKALENVVGARNMDVKPLLNYFQPLFDWLKEQNRNSFVGWNTEWSPYADQSIKVRISLKSALGANAYEWTNNEMFLFRSSVAYAMRKYFSIIKNQTVPFLEEDVRVSDLKPRVSFYFFVTSPQNVSDVIPRSEVEDAIRMSRGRINDVFGLNDNSLEFLGIHPTLEPPYQPPVTIWLIIFGVVMALVVVGIIILIVTGIKGRKKKNETKREENPYDSMDIGKGESNAGFQNSDDAQTSF

SEQ ID NO: 3 - protein, 805 aa, synthetic construct
  REGION 1..805: note = ACE2
MSSSSWLLLSLVAVTTAQSLTEEQAKTFLDKFNHEAEDLSYQSSLASWNYNTNITEENAQKMSEAAAKWSAFYEEQSKLAQMYPLQEIQTPIIKRQLQALQQSGSSALSADKNKQLNTILNTMSTIYSTGKVCNPKNPQECLLLEPGLDEIMATSTDYNSRLWAWEGWRAEVGKQLRPLYEEYVVLKNEMARANNYNDYGDYWRGDYEAEGADGYNYNRNQLIEDVERTFAEIKPLYEHLHAYVRRKLMDTYPSYISPTGCLPAHLLGDMWGRFWTNLYPLTVPFAQKPNIDVTDAMMNQGWDAERIFQEAEKFFVSVGLPHMTQGFWANSMLTEPADGRKVVCHPTAWDLGKGDFRIKMCTKVTMDNFLTAHHEMGHIQYDMAYARQPFLLRNGANEGFHEAVGEIMSLSAATPKHLKSIGLLPSDFQEDSETEINFLLKQALTIVGTLPFTYMLEKWRWMVFRGEIPKEQWMKKWWEMKREIVGVVEPLPHDETYCDPASLFHVSNDYSFIRYYTRTIYQFQFQEALCQAAKYNGSLHKCDISNSTEAGQKLLKMLSLGNSEPWTKALENVVGARNMDVKPLLNYFQPLFDWLKEQNRNSFVGWNTEWSPYADQSIKVRISLKSALGANAYEWTNNEMFLFRSSVAYAMRKYFSIIKNQTVPFLEEDVRVSDLKPRVSFYFFVTSPQNVSDVIPRSEVEDAIRMSRGRINDVFGLNDNSLEFLGIHPTLEPPYQPPVTIWLIIFGVVMALVVVGIIILIVTGIKGRKKKNETKREENPYDSMDIGKGESNAGFQNSDDAQTSF

SEQ ID NO: 4 - DNA, 21 bp, synthetic construct
  misc_feature 1..21: note = target sequence
  source 1..21: mol_type = other DNA
aacaactttaatcaggaagct

SEQ ID NO: 5 - DNA, 21 bp, synthetic construct
  misc_feature 1..21: note = target sequence
  source 1..21: mol_type = other DNA
atcaggaagctgaagacctgt

SEQ ID NO: 6 - DNA, 21 bp, synthetic construct
  misc_feature 1..21: note = target sequence
  source 1..21: mol_type = other DNA
ttgtagtgagaaactttgggc

SEQ ID NO: 7 - DNA, 21 bp, synthetic construct
  misc_feature 1..21: note = target sequence
  source 1..21: mol_type = other DNA
ggcagtcttagactgttcttc

SEQ ID NO: 8 - RNA, 41 nt, synthetic construct
  misc_feature 1..41: note = crRNA
  source 1..41: mol_type = other RNA
taatttctactcttgtagataacaactttaatcaggaagct

SEQ ID NO: 9 - RNA, 41 nt, synthetic construct
  misc_feature 1..41: note = crRNA
  source 1..41: mol_type = other RNA
taatttctactcttgtagatatcaggaagctgaagacctgt

SEQ ID NO: 10 - RNA, 41 nt, synthetic construct
  misc_feature 1..41: note = crRNA
  source 1..41: mol_type = other RNA
taatttctactcttgtagatttgtagtgagaaactttgggc

SEQ ID NO: 11 - RNA, 41 nt, synthetic construct
  misc_feature 1..41: note = crRNA
  source 1..41: mol_type = other RNA
taatttctactcttgtagatggcagtcttagactgttcttc

SEQ ID NO: 12 - DNA, 175 bp, synthetic construct
  misc_feature 1..175: note = donor DNA
  source 1..175: mol_type = other DNA
atgtccagctcctcctggctccttctcagccttgttgctgttactactgctcagtccctcaccgaagaacaggctaagacatttttggacaagttcaaccacgaagctgaagacctgtcttatcagagttcacttgcttcttggaatataatactaacattactgaagaaaatgc

SEQ ID NO: 13 - DNA, 167 bp, synthetic construct
  misc_feature 1..167: note = donor DNA
  source 1..167: mol_type = other DNA
ttatttacttatttttcagagtgaggctgcagccaaatggtctgccttttatgaagaacagtctaagcttgcccagatgtatccactacaagaaattcaaaacccgatcatcaagcgtcaactacaggccttcagcaaagtgggtcttcagcactctcagcagacaa

SEQ ID NO: 14 - DNA, 186 bp, Mus musculus
  source 1..186: mol_type = genomic DNA
atgtccagctcctcctggctccttctcagccttgttgctgttactactgctcagtccctcaccgaggaaaatgccaagacatttttaaacaactttaatcaggaagctgaagacctgtcttatcaaagttcacttgcttcttggaattataatactaacattactgaagaaaatgcccaaaagatg

SEQ ID NO: 15 - DNA, 159 bp, Mus musculus
  source 1..159: mol_type = genomic DNA
agtgaggctgcagccaaatggtctgccttttatgaagaacagtctaagactgcccaaagtttctcactacaagaaatccagactccgatcatcaagcgtcaactacaggcccttcagcaaagtgggtcttcagcactctcagcagacaagaacaaacag

SEQ ID NO: 16 - DNA, 186 bp, synthetic construct
  misc_feature 1..186: note = ACE2
  source 1..186: mol_type = other DNA
atgtccagctcctcctggctccttctcagccttgttgctgttactactgctcagtccctcaccgaagaacaggctaagacatttttggacaagttcaaccacgaagctgaagacctgtcttatcagagttcacttgcttcttggaattataatactaacattactgaagaaaatgcccaaaagatg

SEQ ID NO: 17 - DNA, 178 bp, synthetic construct
  misc_feature 1..178: note = ACE2
  source 1..178: mol_type = other DNA
ttatttacttatttttcagagtgaggctgcagccaaatggtctgccttttatgaagaacagtctaagcttgcccagatgtatccactacaagaaatccagactccgatcatcaagcgtcaactacaggcccttcagcaaagtgggtcttcagcactctcagcagacaagaacaaacag

SEQ ID NO: 18 - DNA, 20 bp, synthetic construct
  misc_feature 1..20: note = Target sequence
  source 1..20: mol_type = other DNA
atctgggacacggagacttc

SEQ ID NO: 19 - DNA, 20 bp, synthetic construct
  misc_feature 1..20: note = Target sequence
  source 1..20: mol_type = other DNA
acacggagacttcaggtagt

SEQ ID NO: 20 - RNA, 36 nt, synthetic construct
  misc_feature 1..36: note = crRNA
  source 1..36: mol_type = other RNA
atctgggacacggagacttcgttttagagctatgct

SEQ ID NO: 21 - RNA, 36 nt, synthetic construct
  misc_feature 1..36: note = crRNA
  source 1..36: mol_type = other RNA
acacggagacttcaggtagtgttttagagctatgct

SEQ ID NO: 22 - RNA, 67 nt, synthetic construct
  modified_base 24: mod_base = OTHER; note = thymine
  misc_feature 1..67: note = crRNA
  source 1..67: mol_type = other RNA
agcatagcaagttaaaataaggctagtccgttatcaacttgaaaaagtggcaccgagtcggtgcttt

SEQ ID NO: 23 - DNA, 186 bp, synthetic construct
  misc_feature 1..186: note = Donor DNA
  source 1..186: mol_type = other DNA
ctgttggccttcctcatatgactcaaggattctgggaaaactctatgctgactgagccagcagatggccggaaagttgtctgccaccccacagcttgggacctagggaagggcgatttcaggtagttgggttgtcacttacagtgcagctactaagtgagaaacagtgtatgaagggttcattata

SEQ ID NO: 24 - DNA, 170 bp, Mus musculus
  source 1..170: mol_type = genomic DNA
ggctgggatgcagaaaggatatttcaagaggcagagaaattctttgtttctgttggccttcctcatatgactcaaggattctgggcaaactctatgctgactgagccagcagatggccggaaagttgtctgccaccccacagcttgggatctgggacacggagacttcag

SEQ ID NO: 25 - DNA, 170 bp, synthetic construct
  misc_feature 1..170: note = ACE2
  source 1..170: mol_type = other DNA
ggctgggatgcagaaaggatatttcaagaggcagagaaattctttgtttctgttggccttcctcatatgactcaaggattctgggcaaactctatgctgactgagccagcagatggccggaaagttgtctgccaccccacagcttgggacctagggaagggcgatttcag

SEQ ID NO: 26 - DNA, 20 bp, synthetic construct
  misc_feature 1..20: note = primer
  source 1..20: mol_type = other DNA
tggatgggatcttggcgcac

SEQ ID NO: 27 - DNA, 21 bp, synthetic construct
  misc_feature 1..21: note = primer
  source 1..21: mol_type = other DNA
aagcaatcaataaccccctgg

SEQ ID NO: 28 - DNA, 22 bp, synthetic construct
  misc_feature 1..22: note = primer
  source 1..22: mol_type = other DNA
aaccttgaccctcagcaaagta

SEQ ID NO: 29 - DNA, 21 bp, synthetic construct
  misc_feature 1..21: note = primer
  source 1..21: mol_type = other DNA
tcaccctacttaggcccagaa

SEQ ID NO: 30 - DNA, 22 bp, synthetic construct
  misc_feature 1..22: note = primer
  source 1..22: mol_type = other DNA
tgtttctgttggccttcctcat

SEQ ID NO: 31 - DNA, 23 bp, synthetic construct
  misc_feature 1..23: note = primer
  source 1..23: mol_type = other DNA
actgtggaccatcttcttttcct

SEQ ID NO: 32 - DNA, 20 bp, synthetic construct
  misc_feature 1..20: note = Primer
  source 1..20: mol_type = other DNA
aagactgcccaaagtttctc

SEQ ID NO: 33 - DNA, 20 bp, synthetic construct
  misc_feature 1..20: note = Primer
  source 1..20: mol_type = other DNA
gctgaccatggtgtagcaga

SEQ ID NO: 34 - DNA, 22 bp, synthetic construct
  misc_feature 1..22: note = Primer
  source 1..22: mol_type = other DNA
tttttggacaagttcaaccacg

SEQ ID NO: 35 - DNA, 24 bp, synthetic construct
  misc_feature 1..24: note = Primer
  source 1..24: mol_type = other DNA
tagccatctgggagagactaggat

SEQ ID NO: 36 - DNA, 23 bp, synthetic construct
  misc_feature 1..23: note = primer
  source 1..23: mol_type = other DNA
gtccagcagcttgtttactgttc

SEQ ID NO: 37 - DNA, 20 bp, synthetic construct
  misc_feature 1..20: note = primer
  source 1..20: mol_type = other DNA
ggcagtcactcatcctcaca

SEQ ID NO: 38 - DNA, 21 bp, synthetic construct
  misc_feature 1..21: note = primer
  source 1..21: mol_type = other DNA
tgaccctcagcaaagtaaagg

SEQ ID NO: 39 - DNA, 22 bp, synthetic construct
  misc_feature 1..22: note = primer
  source 1..22: mol_type = other DNA
acccaagtgtcttctttcaacc

SEQ ID NO: 40 - DNA, 21 bp, synthetic construct
  misc_feature 1..21: note = primer
  source 1..21: mol_type = other DNA
cgtgcaaaagcctacttctct

SEQ ID NO: 41 - DNA, 21 bp, synthetic construct
  misc_feature 1..21: note = primer
  source 1..21: mol_type = other DNA
caaacctttcctaacaggggg

SEQ ID NO: 42 - DNA, 22 bp, synthetic construct
  misc_feature 1..22: note = primer
  source 1..22: mol_type = other DNA
tttttggacaagttcaaccacg

SEQ ID NO: 43 - DNA, 25 bp, synthetic construct
  misc_feature 1..25: note = primer
  source 1..25: mol_type = other DNA
ctggatttcttgtagtggatacatc

SEQ ID NO: 44 - DNA, 29 bp, synthetic construct
  misc_feature 1..29: note = primer
  source 1..29: mol_type = other DNA
ccaagacatttttaaacaactttaatcag

SEQ ID NO: 45 - DNA, 22 bp, synthetic construct
  misc_feature 1..22: note = primer
  source 1..22: mol_type = other DNA
gtctggatttcttgtagtgaga

SEQ ID NO: 46 - DNA, 22 bp, synthetic construct
  misc_feature 1..22: note = primer
  source 1..22: mol_type = other DNA
tgttaccaactgggacgacatg

SEQ ID NO: 47 - DNA, 21 bp, synthetic construct
  misc_feature 1..21: note = primer
  source 1..21: mol_type = other DNA
ggggtgttgaaggtctcaaac

SEQ ID NO: 48 - DNA, 22 bp, synthetic construct
  misc_feature 1..22: note = primer
  source 1..22: mol_type = other DNA
gtgaaatggtcatgtgtggcgg

SEQ ID NO: 49 - DNA, 26 bp, synthetic construct
  misc_feature 1..26: note = primer
  source 1..26: mol_type = other DNA
caaatgttaaaaacactattagcata

SEQ ID NO: 50 - DNA, 25 bp, synthetic construct
  misc_feature 1..25: note = probe
  source 1..25: mol_type = other DNA
caggtggaacctcatcaggagatgc

SEQ ID NO: 51 - DNA, 49077 bp, Mus musculus
  source 1..49077: mol_type = genomic DNA
aggcccatgagccctgccatttaaagtggctcctctcttacactctgggaatgaggacacggagccagctgctgaacttcaccaggataaccattaaaattgctttggagttcatatttccacgatcccatgcctatggatgccaaggacttgtcatggatgcgctttggatttcataatgcagagtcattattacttccttgagttctcagctgagttgtaagcaggtaagtgaaagggaaagaggcacctgataaagtcagctgtaggactagagtttagcatgtctcctgagaaatagaaaatgactgcttgaaactttaccaaagccacacaggaagcatcaaactccctatggagtggagaagagtcttataattttttaaatgggcagagaaatgaatttatttttaatttttagagacagggtttctttgtatagctctagctgtctttgattggtagacaaagctgtcctcaaactcagagatcttccttcctttgtctcctgagtgctgggattaaaggcatggaccaccactgccctgccccattctctccattaattttaagtgaatgcttgcaaaagctcacttctttggtgaacagcttcctttacaaataagtacctttgccttcgtttttataggattcttaaaaagaaaaaaaagattcagccaggtggttgtggtgcacacctttaatcccagcagtcaggaggcagaggaaagcagatctcttgagtttgaggctagcctagtctacagagggagttccaggacagccaaggctacagagaggaactgtctaaaaacaccaagaaagagagaaaggagagagggagaggatggatagcttattgatagaattgtcagaaaaggctataagttccaatatgtgtcccatgatttctaagtctagccctttctgttatagtaaaatcatagtacaccctcctcctccagtgtatctttaacagcttttaaggaacatattaactaaatgtccaggttttgatttggccataaaatgttagcaaagctaaggttttctaggattaatgaataacatgtctttatttagtttacttaaaaaaatcattctaaaatatctgtttacatatctgtcctctccaggattaacttcatattggtccagcagcttgtttactgttctcttctgtttcttcttctgctttttttttcttctcttctcagtgcccaacccaagttcaaaggctgatgagagagaaaaactcatgaagagattttactctagggaaagttgctcagtggatgggatcttggcgcacggggaaagatgtccagctcctcctggctccttctcagccttgttgctgttactactgctcagtccctcaccgaagaacaggctaagacatttttggacaagttcaaccacgaagctgaagacctgtcttatcagagttcacttgcttcttggaattataatactaacattactgaagaaaatgcccaaaagatggtaagttcttgaggctacccagggggttattgattgcttcttaaagatcagaattactgcctataaaactggataaggaaatcatagagatctctcaagtgtgaggatgagtgactgcctctgtagctctgatcctagtctcccagatggctaaattcaattgaccttagagttcatctggaaaattgttatgaatgaattatttgcccagattccaaagatgagtgaaaatgtttaataaagttgccatcactattctcattatatttggtatgtaaagcattcatggaaatgttctaagtcgttattgagccaataattttctttagcttataatgccaacaggtctatccgagaactacaaatgacatattaactgaaaaatgcaactggggtttactgaaggcagcagcttagtaattaaggtaaccatggcttaggtgaaactggacctgggaattccttctttcattgacacagagctctgaggaatttccaaaggtcacagaagaaaagctataattaaactagtcccaaaaaatctcagcctactctgggaaagcagcatattttgtttgacaagtgcaaggacttagaacttttttttttctcactgatcctgaagtgccttttaagtatagttaagtggtggaaaattgagcaactatttaagaaaagactcttttttttcttcttccagcaatgctttccttcaaaacggtagcttcaaaacttcctgtcttttaaatgatcagggggctgtgtgtttaaattattgccattcatagaacagagtgggtctgaggatgcctgtttcctttgaaattctatgccccctcccagttttctaaaatttaagaaaccacagagactttgacaatgtagttgccaaatgagttgcttttaactgctctaatagtttggtcttaccggtgttgtttttagtgtaactttttttcccctcagtttttattttatttctgttggagatcaccaacgtgtactgtagattttctgagctgaatggttcaaggtgagactaatattatacttgaaagcctagcctggagctgacatccagatcctggctgtaccactttttagctgggtaaacttgaattcattaaatggacagtctgggtcttagttactttttgttttaagatgtattttaattttaaaactatgtgtatgtgtgtgtgctcccgaatataagtatagatgcccatgaaggtcagagatattggaacccccaggagtggtgcagcaggctattgtgtgccaccagacctagatgctggcaacttgcaacctctgcaagggaagtacatatccttaaccatcgagccacctttctaccccagtcttagttgaaaaataacactcacatatacttcatcgagtatacaaagataccagaagaatgtgtgatgcttgtagggttaggagaaaatgaagatatcatctttattagtgttgaaaggagttagggtttttgaaaaggtacataatagctctttaaaggacctacatatacagaagtcctattatagagtgagatgtaatgctgtttgctgaaaccatctctgagattattatgatgttggggtttggcttagtgcaaaaaaaaagagagagagagagagaatgttaccatttttttaaacagatttatttatttatttatttatttatttattatatgtaagtacattgtagctgtcttcagacactccagaagagagcatcagatttcgttacggatggttgtgagccaccatgtggttgctgggatttgaactcaggacctttggaagagcagtcggtgctcttaaccgctgagccatctcaccagtccgaatgttatcatttttaattatacaggttgattagagcaaaactagaaatgggagagtatgaatgatcagctgttttaaaccggaaaaaagtgtgcagcctttaaagtgaaaaaaaaaaaaaagaacatttaagtgaaagaaaaatgacaagggcctccatagcccccttcaccagagtggtcaatggtgcactaaagcgcttaggtgaactggtgtgagcgtgcctaaagagggaggcacttgtgggggaggaagatggtccctcttcccattctctcctagttttatgtgttgcaattctcttatatgctaggcagtggccaggcattagaggaggttactgaaatatgctgtgctataagggcaacgaattgacacggaatgtgaccatcacatagtgagtacaaagttgggacaaacaacctgggttcagaccctcctttgaatgttagaagctgaaggtcttgattaagtgccttattgacatcgatttttttattgtctcacatactatatcctgattgcagtttgccctccacttctcccagttcctctccattcctcctctcatccggatccaccccctttctgtctctcgttagaaaacaaacagacttctgaaagataataataaaatgaaatgtaataagatagaacaaaaactaacgtatcagattaggacaaaacaaacaaaagaaagagcccaagaaaaaggacaaaaccccagatacagccgcagagacagtcattcatacactcagcaatctcataaaaacactaacctggaagctgtaatatatacgcaaaggacctttaggagggaggaggatgtatataaaataaaaaagaagatgaaattaaaatgcaaacaaacaagcaaaacaaaacacaaaagttatccctgcaaagatgctatagaggtttttgtttttgtttttgtttttttccttcttggtcatctactgctgaatgtgccgcctacccttaagagtagtttatttccccagtgtgtctcctttggaggaaagtaaattttcatctgcaagtggttatcaattggagatggatggttagggatgggggcatgcgtccacttctttcatctctaggaccccgtctggtgcagacctgtgcaggccctgtgcttgctgcctcagtctctgtgagttcgtgtgagctttgcttgtgttcattcattgggccttgtttcctaggtgtcctccaccctctctggctcttactctttctgcatcctctcccacagggttccctgggccctggtggggaggggtttgatggagacagctcaactagggctgagtgttccaaggtctctcattctgcacactgtctggctgtgggtctctgtatttgttcccatccttcagacccatttaactcagatgtaaactgaataaaattatgatcacggtattaatagtagagtatgtactatatggaaatgctggatttagaaaatatactctgtgtgtgtgtgtaattggatactgtacctggcattagtttgaccttcatcaagattattattcagtgggtttacattttgaaaatccaactgtctcactggtgtctccagtatggggtttttgtttttagactttttggtgtgtggtacagggatgtagcccagaaccttgagagtgccagctaagtgctcagcattgagctccctgctgagcccaggtgcttttgaatacagatttcccctgtaaaggtcaggtttgaaggaagctctcaggctcttctgatattcacagaacagctgctgggaagtaagtttctgggaaaaagccatcgtgttccttcctttgcctgcctcttggccttgtcagtggagaattcagggtcaagaattaagaaatatcaacctttgtctgtatattttcacagtaagcctttactgggcaaaggggagaatgtgtccccataaatttgcattatagtgaccttactcagttttattgtggatcaacacttgttcttagatatgcactgagttgcagaattagactgtgagtttgcttgaataagattatctgaggatccagtttatcttttcgggagttaatggaaaatgaacttcttgaatagataaaagaaatagttctggaattcccagagtttaatatgtgctcagcagtaactggcccgtaactgaaaacaagtgaaagacttgcaattaccactgatgcatttgaggtcgttaggtttttctgatcttagtttcatgatgcatttattgctttagggagagtatacaagttttgaaaaaagttctcccatgaaataaatatttacaagatgcccaaagaaagtttatagatcactagcacattggcaagtatttgataagactaatcctaactgtaacaaaaacatttaccaccaaaagttgtcatatattatggaaagacataaattgaaattaggctggaaaatacaaattgtgtataagttagttcttctagggagcaactgaggcataagaaatagtttctgatcagaagagagcctagacgtcagtttggattgtctcctgaagagattaactaaatacatttttctgatgacagtgattgccgttgttcagagacagttctgggcgataaaatgagaacattattctaagccttaaaaaacataatgccggagtaaagatgtaaagaagaaaagaagagaaaataacaagaaatggattcttgctccccacccccaccccccaacgaataatcagaatgagttaagggggtcagagagggctgggaaaagtgtgacctggaacaagaaaggagaggatgttgacagttgggttggctcagtctttttccacctgaaaggcaatacagccaaaagcacagcagagataaattcagagtaaatagagggaagcactctttgcactgcaggtggtgaagctgataagacctgtaaaactttaatctaaaaaatgtgagacattgacaactccaagtaggctttcaaagcatttaaacaaacatgtggcttgccgctctactgtgggctaaggaagtatggtcaattgcattgatggactgtagacttcatcctttcctaggatgatatctaaggtcatgtcaccgtgcagtgccatcccagtgcaggttctgggagtgtgtccttgatcatttgtcctgatttctgtgaagcaaggctgctgacaactgacttagtgggcttgaacaatgaggttcatccttcccacttagggatttgccaagacaacctgctcaggttagccaattgtaagcgagtgagtgcgagagagacttggagtagttgcccaagcagaagccaactttcagagttgcatgcaaatgcagccaagatcaacagaggtgtctgccaactccaagctattttaggcacataaacaataaacactcatgctattgacgcttttcgttgtttcttagatagtattattattgtaatagataattgatacaggacaggaaattagactgtatgacatttctgtgttcatgtcctatcatggtgacatttctgaggtaacattcaggagtcaggatgttggggttaccagcctacagatctagcatagtatggtgttacttaaagttgtgagaacatgcctgaaaataaaagaaagccacaacccttttccatatgatgtcctaaaaggtgcttacccaaagggaggcagaaaccaggaaacagggataagaaaaaaaaaaaacttggctcaaaacagctttttaaagctctctacttttctctgctatggtggtgtttgtggctaactccagctaaactcttgtcttcttatgagacttttgagattatattatttctggccaagaaataaaatctagaatcactttattccctagtggatttggatgaaaactggtaaaaaaaaaaaaaaagttcagctataattccaatgggaggcattggagaataaccaagctgggatgctgggtctctgagtatcttgattctctggctatgataaaaactgaccaaaatcaacatggggaggaaagggtttatttcatctgacttgtttgggtaacagtccatcactgaggggaagtcagggcagaggaaactcaagggaggaagctggaagcaggaactgaagcagaagtcacaaggtatgttgtttactggcttgctcccggtgacttgctaaggttgctttcttagacacctcaagaccacctaccccgagacaggacaacagcacccacggacttgactataggccagtctgatggaggcatttactcagttgatcttccttcatcctagatggccctagcttctatcaagttgacaaaagtctaaccaggacaataattgatgacacacacgtgttagctatattcagctcactatcacttaacttctctagctgaaaaggtgatcactatctgtacaattggaaatgcttcccagaaaaggcaatttttctatttccatttatgcctctatactgataggttagttcaacagttgatatgtaaaagctttggttaaaaaaaaaaaagccgggcgtggtggcgtacgcctttaatcccagcacttgggaggcagaggcaggtggatttctgagttctaggccagcctggtctacagagttccaggacagccggaggtacacagagaaaccctgtctcggtgtctgggggggggggggggaggtttgcaagtagctcagaaattcagtgaaaattccttacaaactcatgtaatagactgcacaacaaatcttcacaagaaatgtagactgagtttccagcagctcaagtgtaagaaacttctcaggaaaatggccctacttcttttttaaggcattacccattcttcgtagtttatggtggtttttgatttttgaaataagattctttgtaaatcttcctcaagggcacaagtatagttatgagtgtgcatatgcttgtgtgagtgtgtgtgtttgtgtgccgagaggttaaccctgggtaccattgcccagggtctagccacgttatttttgagacaggatcttcttctgaacctagagctcactatttgattagactacttgactaaaaagcccaaggagcttcctggcttcatctcccctcttctgaaattacagatgagtctcactatacctgactccccccaccaccaccttttattggatattttctttatttacatttcaaatgttatcctctttgcagggctccccttcggagccctcctatcccattccccctactcctgcttctaagagggtactcccccacccatccatccactcctgtcttcctgccctggcattcccctactctggagcatcgaacaccctcaggcccaagggcctctcctcccattgatgtcaacaaggccattctctgccacatatgtggccggagccattggtctctccatatgtactctttggttggtggtccagtccccaggagctctggggggggggtgtctagcctgttgacactgttgctcccactatggggctgcaaaccacctcagctccttcagtcctctctccaactcccccatctgggaccctgtgctcagtccaatggttggctgcaagcatctgcctctgtatgcctgactttttaatgtggtttctggggattacagccaggtcctgatgcttgcatggcaagcacgttgctttctaagtcatatcctgggcccttaaaaatcctttttatttttatttcccacacacaaagatgttttagggaaaaattggggccctcctaacaacaatttcaaaacagtcaatggttggtttttaaataaaaaggttggtagctctccagcagtatagaatgtctctggatcatttgaatcctagagagagcaagaggaccccagtatttagcaataactatggcaaccactcctcactccggtccatgtccctgaaaagcactttgaagattgtcttgcaactattttatttaaatcagaagttgtttggagagagtaggaagctggtgtgtattctcttggtgatctctggggcccttcagcgtttctgctgggtcctttagaggatctaggcatacccattaccctttcctatttctgccccaggggaactcaggacctgataatgctaaataagctgcgatcctccatccaagaaaagaatgttgacccctgcctctaatggttccacaccaggcttcacgacatgtgtaataaatacccttatctgagagcatgagtaggggagcatcagcatggtacaaggataccagaattcagggctgagtattttttcccaaaagataagagacaggaaaataaaaatgttttcaggagtcgaaaagcttaaaatgtcaacaatctcatgcaaaatatagaaaggttttggagttctaggatggagtgacaggattcatataacttgtgttacgtacttcgttgaatagaataaatttctttttcatttaaaaatctctggatgtgttgaaaaagtgatcaaggcagctttgtcctctgagcatcatgcagccttgagggtcgcatctggaaaccagagcagcctatggggcttgagggtagagctttgccgaggaggtggtacaagtgtcacgtcccagatggatgctcaggttcttgcagttttatttttcagaaaattgccacaggcttagggctaggaggttgcagatggtttagcttcattcaggggatgatgatctctagaaaaagagaaaataaagtgccagcactttatatctcaagcaaattatcagaaggagaactggcactcctttgttggccaatgtgttttcaatagaatagttgcatattttagcatataaatacttcacaggcattaaaaagtatgatagttatctatcaaaagcagggatcaacaacctttctagttaagtgttgagctgtgggccttcttggcttcgaaggttttatggtttcttcagaactaccgagccctgcagttgtgtggcagaagcagccataatcagtgtggtataaatggatgtgtggctgaccaataaaactgaatttaagaaagagcaggtgatatctaagcttggcccaagggctataatttatcagccctgattaaaggaagaagccctgatttataattttgaaaagaaataaatataggtcataaagagttgcctaggaaataagaagtttatatagcactaattctatataattatgttaatgacatggatagctagatttgtttttcatcactacagaatatagtgggttttttttcttcactatcttttaaattgttaaatgataacatgcaaagcaaggagattcattgtagtgttttcatgtacactttgcttataatcagtgcccactttcttacctgttctcctgctccttcttgctagtgtctgtcttacccccctccccccatggcagctttctgcctttgtgtcataccctttctctttttcactttcctcttttgccctcttttaaaatggtttttcctttgtcatgatcccctttttagttttcacaaacacagagagagagacagagagacagagaccgagacagaaagacagatagagagagaaatagacagagacagagatagagaaacacacacacagatcgaaagatctaggtctacacaaaagaaaatatgtgtatttatcaatattattgtttaaataaaataaccacaatttgggtagggccgctagacctaaggtctaagacaaagctctcatgaagacgtttaagacgataagacatactacacacatagttagaattttgtttttgaacatttaaaagtagaatttactagtttatacttgtagtacttggatttgccaggaagtctcatggggtggacaatacagtattctctgcagttatagatggaagtcagagagtgaatgtcttcctggttgttttaaaaaatatgtgtcttgtccccaaacctctacggatggctgtaaaaaaattggctgttttagttgctacttgtctgcctttgcacaaagggagtgtggaaagctacttacttcatctgagcttgctcctttaagatgaggagctatctaagcttctgtctaagctttacttcatacattagccaagtccgtaattcacatggagccatcagccacggtctaggcatatatgaactggatgtatatgaactagttttgcatctctgtgtaacagctaagctatttcatttaggcgtgggagccttgaagatatggaatacactgtaaaataaatgcaaaagaatagcttgctagtcaaggtaattttaaatctcataatgtataacacactcaccacaacacacacacacacacacacacacacacacacacagagcctataaacatttgttccacagtttctgtgggccaggggtccaggcatgcttcttctgtttctaggtctcatcaggatgcgttatctagagctgtgttttcatctgaggcttggattgggtccaatctcactgctaagtcaggtgtggccagggttaacattgagatctttgcatactgccaaattgaggttcttggtttattgcagcatattagtcagtggctagtatcagtttcttgccatgtagccctatccatggggtaattagtgaagccaaataaaataaccagtctgagtctcatttatttgtaagcaagtcagggataagcttaaattgcttgtactacaattaaaggcgattattagaaccatgaagtatgtagtttctaacttggtatcttgctgcattatagtccacaaaagagagggtctgctaacaagataaaaatgagtcttacataatgtaaatctacacatcttgctacttttgttccatgtgattggttagaagcacgtcacaagtctagtctcactgaagaagatatcacacaggggtttgactattagcaggtaggcatcctgtaagccatcacagcatctatctaccataggttctagaatagagtgctgggttgactatcaggttcattaccttgctaagcctcttggttaatacataaaaatggtgacacggtgattttccaccacatggggttgtttaaagctcaaaagaggcaaattgtttttcaaagcattctacagtcaccccaactcctatctacttcaagattactttagtcttatctcctctatgaacatttgaacacatttggacccctattggattttttactttcttagtatggaaacgtattgactcatttgtggtacctatgttattatatatttgtgttgcattgagaaagaagaattccagacatgtatcctactcctttgataaaattctaagcaacttaataacaaacatttttttctctctaagcaataaaatggcatttatggagtacttaggatgtgccaggcctgctggtgagcacttcactctttggtacatgtcctaatccttacgacaattctatagtcacaagtcatgttgttaggattgtttaatcttgtaacagaaccacagctagggaagattatgtgtccaaagtcacacagctagccagaactgtctgactccaggagtggagctcacataccacctctgttgtatggcccccaaacaatatgatgtacaaggttccttttactctttgagacatggaggcgacgttgtgcattgactgttctagaaaagccaggagaagtctttccttctacttattatcaagatggggctgcagggaggcaggtgtatctcagcatcatcacctgtttgtagcatcatcacctgtttgtcgacttctttggctgtcagaaaatcatcactctaccaagtggcctggttccaggaagccttcctttgagatcacaatttacggccaaatttcctgccctaaaccatggcacctaggagctttctttgatctggttctcatttctcatggctccaatggtcacaagggactgtattcccattgtgtctgtcgactctgagatctgcaaagttctccttttaggccattcatccctgaatctttagccaacttacccacgtcacagcactgaggccataagttccaaggcctcacagtgtgaattatctgtgagaatttacttccagttcttgggacaggacccttgggaaaactaggcactgggccagaaagaaagtggcccaagagtggcctgattacttttaacctaaaccttgaccctcagcaaagtaaaggactaagccatgcaggaagtagtagatgtttcaccagatgttctttggactatatcttctatccaacttcaatttatttacttatttttcagagtgaggctgcagccaaatggtctgccttttatgaagaacagtctaagcttgcccagatgtatccactacaagaaatccagactccgatcatcaagcgtcaactacaggcccttcagcaaagtgggtcttcagcactctcagcagacaagaacaaacaggtgtgtttgtaaacactctgggcctgatctcaagagttgaaaggtttgagaagtaatacacattcatatcccatgtgaatgactcctttctttgcaaagttagtaatgccagttctgggcctaagtagggtgactaaaaaatctataattcaagttggagtctttttgaggttgaaagaagacacttgggttctgatctgcacagaactgaatagtatatgtctgctacaccatggtcagcactgactagagcaactttcttttttgtttgttcttattgctaagtgccatggttagttcatattaacctcactgttaatgaagttcccgaggttattgtaccaacacagcagcaaaatggcacttaaaaaaacttcctgtgatgaggtgatttcaagaattcttatatttaccctgttaaaataccatctctcatcccatgcccccactccaggggccaggaaacttgaaagaaagctgtaaagagacataaaataaatatcttaaggttttcaggccatattgtctatgtcacaaaaaggcagctctgccatcagagcaggaagtcagccatagataatacatagatgaagggatgtggctgtcttccaacaaggctttataaaaaacacacagcacatccctgatatagcctattgaaatatttttatctaaaacactcagctctttcacatcaaagttttctttctcagttgcttggcctctggaactctgtatgtctcaatctgcatttataaaaataagagcccatgataagatcagagataaagccatatggtatttgttttacaagttgatattttattattgtacattccttggatttaattattgcactaagttattaatctaccttatcttgccttgtccatgcttcttcatctattttttttaaaggattccatgagaaatcctggacgtgctacttttaaaatagcccccaattaattctaatgggtagctgcaattgaaaaacacttaagcgcaggtgaaaacttgaattgattgagaacagaatggtgttcgggatgcatggagaggaaggaggggggattgaaataggtagataaagagaccgtgaagaattctgtgccaaggtagatgtattggattagcctatgggatcttggggaagttgtatgcatggagtttatcttatgttttctaaacaatatgtgtgcaacagggtgaaataataaataataatagtaataataataataatacatgcgggtgttagtagaaatctagcattgaaatgatttgtatgaaagaatcagccttttttcttcctttctctctttgtgcctccctccttttctctcctttcttcctcttttcttcttatgtttccaaactataactctcacagatcaacacgtgggtaagttcattggtttcatgcttaaatatcatggatctattgaattttccaaaaaaaaaaaaaaaaaaaaaaaaaaaggtttcccaacatttcccctttccacccctgtattttatctcggagtggaaatgaaccttttgctgagctgcacctattgacgcccttgaatagcttggctgtaccaggttatcttcactcccctggacccacagggtaaagtttaaattctttatggtggcatagattgccacttgtgatctggatctcacttatctcttcgttctcatggactccattccttcagtcgctggagatgtagtcatttcacatcttgttaaaaatggcttcccttcctcacagaagcagggtaaggggggatgggatgggggtttgcagatgggaaatcaggaaaggggataacatttgaaatgtaacaaataaaatatccaattaaaaaaaacatttaaaaaatggcttccctttcacacctttgtccattcttggggtccaccttctccttccctttatgtgaccagctcttaagagtcctttctctctgaagccatttctaaatctccaggttatgagtaccccttcttccaccctcccacactccttaggcagtactgctacatcactcattggtgacatttaacagtacccatcactcctttatgttgagatccattttgtggctgccttattagtccatgactttggggtctttgaaggttagaggtttgtttgtttcgaggttatcaggatgtagaacatagtggaggcttagtgaattctagatgaaaccaaagtgcaatctctgtgcctatatgagacatgatcctgggagtttgggcagacatgtttattttcattaatctctgtgtttatcacacactctcaagattctgtgatagttgacagcagagacgattacacataggaacatgcttggcaaagaagatatggattgtatattttgaggcatttaaaaatcccatgaccatatgagctcatagagaaagagggagcacgcagaaggcctacatggctctataccaaggcctctttgtatatacaacaacttttagcttacagttttaatgggcatcctgactatgagaatgagtgggtctctgactcttgtgcctggtcctgggactctttccctcctgttggattgccatatccaacttcaatgatagcttttgcttcctattagtatatcttattttgtcttgcttggttgttatttctaagaagcctgctttttttttctaatgagagacacaaaaggggatgattccagtgggaaggggaggtggggaggaactgggaaaagtagaagaggaaactataatcagtatatattatatgagaaaataatttattttaggtaaaatgaaaatgagaataagaaaaaatcccatggggttggagagatggctcagtggttaagagcactggccactcttctggaggacccaggttcaatctccagcagccacatggcagctcacaactgtgtgtaactccagttccagaggatctgacatacctacgccaatactcatgaaattaaaactaaataaattatttttaaaatcccatgacaaaagaatatctttatgccactacccatcatagtacatttttctttctttctttctttcttcttctttttttttttttttttttttttttggtttttcgagacagggtttctctgtatcaccctggctgtcctggaaaacactttgtagaccaggctggcgtcgaactcagaaatccacctgcctctgtctcccgagtgttgggattaaaggcatgcgccaccacacccagctatagtacattttttatgcctccgtctacttattatgtttaatttgcagttgaacacaattctgaacaccatgagcaccatttacagtactggaaaagtttgcaacccaaagaacccacaagaatgcttattacttgagccaggtaggttttcaggaggtattgttttcagtagtcgttacgaaatttatattttcttccaaactgtaaaaatgagatagacttttaaatgtgtgatatatgtgcatgtttgtgtgtatctacatacatgttcatgtatgggcaggtatacatgtttgtgcaagtgcacgtggatatagaggtcagaggacaactgccattcctcatgcctcagaaaccattaacaggttttctttgaggcagtgtctctccctggatgggaactcaccaagtaggtattattgttggccagtaagcccaagggatttgcaagtgtgtgtcctctgggctggaattagaagtgcatatataccaccacatctgatatttagtgtgagttctgagacctgaactcaggccctcgtgcttgtacaagcactttctggactgagccatttccccagcctgagacagacttttgacaacacaatgttttgcagatggacaaccttaaaatgctactgttccaaagggttccagctcacacattaccctttttaaagctctgtgtttatttttcattgtatgtgtgtgtgtgtaggggaagagagagagagagagagtaagtaagtgcatgtgcaggcacgtatgcctatgtaggaggtcaaagaaaggtgccctcctccatcactctctgcctttattcccttgaggcaagcctctcactgaacttggagttaaactggcagccaacgagccccaacaatcctaatgtgtatgtcctcatagcatgggggtttacagttgcatgcatggtcatgcccagacttgtttgtttttgagactataacttacatttcttctttctctttctttcctttaacccctgcccactttcctcttttacatggcgtgtgggaatttagactcaggcccttgtgtttgtgagcaagcattcttattccctgagccatatctacagtctttcatagatatcctttaacttaaaaaaaaaaaacaaaacaacacactgtaaattttattacattcaagtaatttaattaagctagtaactaaataacttccgtgccctcctgccctttgcagacgcatttgggtctaataataagtccaaggagactgaccatgacattttgttaaatggcaaggatgcctgccaacctttgaattctttaaagtctatgtaattgtaaatgcaaattatttgtacgataaatacaaatgtttcacctttgtgtttattttcctctggaacaacaggattggatgaaataatggcgacaagcacagactacaactctaggctctgggcatgggagggctggagggctgaggttggcaagcagctgaggccgttgtatgaagagtatgtggtcctgaaaaacgagatggcaagagcaaacagtaagtccgctgccttgatgctctccatggggggtggttatgggactatttctcattcataatcaagcttgtacctataatattttgatgcccctcaatcaacatcatttgcttaaaggttcaggttaactgtcccaaaaagatactgggacattttaaaaaaatggtacactggaactctattgttcaaagagctttatggatgagattcaactatgggatgaattatctaggacttatgaattactaattattcatggaagtctaaggccccaaagggtgcccatgagaatgttggcactgaatggtgatcctatttttcagatcagataagagaaaaagacaatatccacttaccaaaaccctcattttgtcacagtttattctccaagttcatttgtgtgtctgcctcagggagatagctcagaaagaagacactatcaattagttcaggtgaaaattatactttttgatattctaggtcataagaatgttaagatgggaagagactttcatgacttttaagttctcttggaacaatgagcattatgaataacttcatgccttagtggttcactgtgccctgggaaataaggaaaaacgtggtttttttcatttgttatttctaatttcttgtcatttaaactcatatttcctcaagccttaagtaagttttagaccatagcttatcatccattttaataatatccatgtatctttccatccaaatatttatttttctcatccctttggtcatatattaacaacatagatataaaaacaaaattcattaaaaacattcacaaatcttattagctactgcttcaacacaagcttgtggtttttgatggttagatactaaccaaaacacagagattggctttgtatagataacatttgcaaacaaacacaggaccactgcaatagtgtttgagatgaagttggtggcatctgaaataggccctgaagaaaagcaggattcaaaacttagggaccaattgcaccttttaaaaataataatgcagagtatgctccaagggtttggaaattaggagtacatttctgccccttgccagctgtgtgactttggacaagtgattttactgctctaagcataggctgttgatacaaatgtagataggtgtagccattatggaaaatggtctggcaggttctacagacattttagatagaactactatatgatctgctaattcctcttctgggcatgtactgagatatctgcattcatcacagcattgtaaaagatagtcacaacagggaaacacatgatgtgtccattaacagacagatggataaagaaaccacagaatctgtatatatatgcatatgataaatgatatacactatataatagaatatcatttagctttaaaaaattgccatttgccataatgtggatgaacctagatggtagtatgctaagtgagataaatgcaacacaaagaataactacagcacatgctctaacttaaatagagaacctttaagtaaaaggatcagctacctatacagaaaaagaacaaaactgccattaccaggggtgataggaggaaacaggaaaatacaaaccaaaatattctaagtagctaccaaactaagaaactactaaactaaaccactcaatgggtagaaggaaaagtttattgggaatcaaactgccaaggccatctctggaagtgggaccgatagaagagcaaaatgacagaaatgcaagcagaccttatacacaagtcagcaaggtgaaatcttgtcagcaaggctgtccagattggcggttaactaggtacacttgtccaaggtagttgacctttggggtaagactaaggggtcaggggggctcctgaaggattaaaggtggttcctagcaaacaggaagtcactttaggaaatctgcttttctggtgaacaggaactgcctttaggcttgtttactcggtaacaatccttctgtttacctggctgaatgaagtccttcagtttagggcaatgtcaccaacactgccattttgggaaaaaggggaattttagacttaactatcacacactcaatatgaggactacagtgaaaaatagcctactgtactcaggatttccccccaaatgtgtacattatcattgctcttgtcttaaggcaaaaaaagggtacctatgtaagatgttcatttgtttccctgtgataagccttatgctgcataggtgacgtaacatcatgttttatgacctgaagaatgtacggaaatttttatttctaaaaacaggctgtacaggtctcatgcagacagctacagtgttgtaggatcatgagtgtgcagaatgttcagcaatcacggccctccctatctcgggcttttacaatgtattccttcccaaccttctgatctgggaggatcagggaaaggaggaggggtgtggatatgataaaatgcattgtgtgaaaatttaaaggaattaagaatatatttaaaaagcatgggttgtcatttagtacaatgggctttaaactggctacactataccatggcttctgtgcttattaaaccagaaatttatttaaactcctgatgaagatgaaggacagttgcttaacagaattagcaagtattgcctttcatttgggttcttttgagatgacatgtagaagggcaacagaaatccttaagatcattttttaaaaattctcccgtcaagattaatctgtgtaaactatatattttctctttttatcccttaaaaaacagattataacgactatggggattattggagaggggactatgaagcagagggagcagatggctacaactataaccgtaaccagttgattgaagatgtagaacgtaccttcgcagaggtaatagagggactgcaggacaataaacaggggccataatagccaatagacaaagactacattttcttttctgttttagttctatcatatattacatcccactgcagtttcccctccctcctctgtcccagttccccctacatctttctccccaagatctacccccacacacaccttagaaaggagcagtattcccagggacatcaaccaaacactgtgtaacaaagtagcttttttttaaagacaggttttctatacataaccctgctatccggggactcactgtgtagatcagactggcttcaaactcagagatctgtctgcttctgcctcccaaatgctgggatcaaagcatcacgaccagtgaaggctattgttttaaacatgcaaatttgccgtttccagttctcacagaactatagtatattagtgtttgtaaaatttctttttttgaggcagggttttatgtatcctaggatggcctctaacttgccatgaaactaggcatgaccttaaattcctgatcgcttctgctttcaccttcttagttgggattataggtgtgcaacaccgtgcctgatttatacaaggctggggatcaaactaagttttggtgcatgcttggcaagtgctctactaactgagctacatcctcagctttggaaaaatatttttcagtaaaatctgtacagctcccttagacaatcggaacagcactgtttccctctcttttgctcaattaatagtaattaccaacaggaagacaataaaaaggaaagatttagatcgtgagcaatcagcctagtcttgctttcttagcagaaagaccatggctttgctttcttcctcacaactattaagattattgtttcatcagtctttgacaatggaagtgtatttgtttctgcctctcccctttattccttccctccctccttccttctttccctccctgcttttctcctccttcctacttccttccttccttgcttcctcccttgcttcctccccctcccttcctttctctttctctctctttctttctttctttctttctttctttctttctttctttctttctttctttctttctttctctctctctctctctctctctctctttctctcttgattactagaaagagtcctctgacaaaagaagtcagtaacatctcagaatgatttatttttttggcatcagcttttctctggggccttgcggtcctccaagctctcatactgatttatgcagtggggctagtcagagtcaatatcagttaagaaagaatgctcccaaactcttcagaatgtttgagaggagaagaaactgtcctgtatgaagacattcttatttctagagttgcatattggaagctgaagtattcttggtctaaaacttttcagatcaagccattgtatgagcatcttcatgcctatgtgaggaggaagttgatggatacctacccttcctacatcagccccactggatgcctccctgcccatttgcttggtaaggaactgcaggagccctttatgctctttgttgttataattatttccatgactcaaactgtcttttggagcgaggctcaatctaaagcatttgagaatgtttgaaaatagcattattatcattacagaaatatttttaaaagcacattttcagcagaaaaagtccatttaaaatcattatcttatctatgctatttaaaatgaatgtagactttaaatctatcctctattaagaggactagaggaagaattctagcttaaaatgtatttatccaagttggttgactaagcctgggattttggtattatatggtcaaagggaaacagctcagcgggacactgaggaggacattgtgtaccggatggacttgacttttgtagtgaatacttataagtgtaactttaggacttcacatttatctctagtaaattgaaatagcaaaatggtgtggtgggtaaaagtgcttatctgcagaaaccatgatggaaagagagaaccattgctgaaaatcgtcctctgacaattgctcacatgcctccccatgtggactgtggtacatgtgcacacatgacacacaagaatgcatgcacacatgtacacacacaattattattattgcttattaataattggattcactggagtgctttccatgtaattgtgtggttccatattttcagtgccaggcttttgattctttgttattatagtttgtttgttttttttctggatccatctccctttttaggatctctggtcttgaattcaacttcacattgttggatatgctcagctacctagttaactgtgttattaaggtattaaaagtaaaacttagccatcagttaatagaaacttttttttttcaagacagggtttctctgtatagccctggctgtcctggaactcactttgtagaccaggctgacctcgaactcagaaatccgcctgcctctgcctcctgagtactgggatcaaaggtgtgcatcaccacacctggcaaatagaaactttaatgatgaacttatgtgatctcttttgaatccacactagaccttactaatcattttctttgcatttgcaacattttgttgtgtgtatgatggctttttacaaacctaatgtgtagtagctatgttgttccatatgttttattttgttttgctttgtttgagacagggtctcattgtacatcccatgtcctgagcttactggcacgcactaccatgtccagttttaattgtgtcttagtaattcacaatgggagaagctctgaagattccctagcaagctttggctaaccacataattttttgtacagttagtattgtgcatgtgatggagagggaaatgttgcaaagttaacataagagaagaactaaaaggagaagcaggagtggtggaaagggaatgcagaatccagactgtttacaaatactgaggggctgctttgcccagccaacctcatctatgaaagattggatctgtattttggttgctttactcaaatattaatgtattcttttcgtaaacaggtgatatgtggggtagattttggacaaatctgtaccctttgactgttccctttgcacagaaaccaaacatagatgttactgatgcaatgatgaatcaggtaggaaaaagaagccttaaaacaatgagatgtttcaactgcatttttcttttctgcatctctctgttctgatcttgtgtatgaaatcatgctgataaaatccaagctaacttaaacttatttcttatgtattttcaatgaagacgtagtttataatgtcaatgatctcaagtaacttcaataacagaaactcttgtttgttttgttgagaggcattcaaaattgagatgacctggtttgtttttttttcctataagcaataagatatggatttatcagctggctggcatcaggtttttcaaaatcaatttttactaagtaatcagtctgtaatctctatggatagtgatgaacagaaacatcttattttggatattaattgccattgaaatgttaccaaatattactttaattttgttaatattactattttattactatttaatattaaatcctttaaataacaccctttttaccatttttatataattgtttaaattgtgttttcctccattaatgaaattatattagaatcttagatatttttaaattttaattaagttctagatttctgattatgagacacttccatttgtctctaaagaatgtcttcagatggccacacaataattgtgcatgcctgctgaaggtattttctcattaatccctttacatctgtaagttgtaatgcagcaatgaccttcacatatgtaacatactcctgggaaattttcacagtcaaataaaaccccaaagctgcaaaacatcaattcgtgtaactcttctaggtccattttgttattgcatagcagcagatggccatcatgatgctacaaacctttatcagtggcttcatgtatgttgtatagtgataagcatagcagccttccaaactgactatgtagttgctgttttccttaatattaattctgaaagtgtcaggcttggtatcatgatagttccatgctataagacacaaaacaggcttagatggatgatgcacctgaagtcacaaagatagtcagtcagaggtaactaactcctctaatgtttctcttctacttcaagatacgttgactatgcttaccagtctgtgctgttttgttcattcaacataagcaatactatattaaagaacaatcttgagatgtatatgatattgaaagctcccatccctaataaccaaagcgattttgaccacaaagaacaaaggaagagatgtgacagtttctgacttcaaaacacactaaaaggaagaaacactaaaaatagacacacagaccattggaacaaaatagaaagcctggaagcaaacctacacattcaaagccaactgattttcaacaaagatgctaacatgtcaatatggaaggcgttacattagagcaaggacagcctatttaataactactcaatgctgggaaaaattaggtgtcctcatgtagaagaaccaaataggacctttgcctcttaccagttccaataattaattcaacatggacaaaagatgtagctatgagacctgagactatgaaacaggggaatcattccagacattggaatggataaggaatttttgggcaaaacttcccaaatgcagtaatcaaaaactaaaatagacaaacagaatttcatgaaagtaaaagacttctgcacagcagagaaaacaacatacagaacacaagaaaatgtttaaaatgtgcatatgcaatgagagttaatgtctagaatgtatacggaatacagcagcaaaactccatctaaactgattaaaacatgagcaatagatattgcttgaaagaagccacacaaatggttagtttttctataaaatttgtcaacaccaccaatgatcatgaaaatggaagtcaaaacaaaaatgcaagacgacctcagtccagttaaaatggctatttaaaaaaagacaaaaatcccattgagtgctggtgaggattcggagagaaccttccacactaacttgggaatgcaacttagtacagtcatgagagaaaacagccacttccttgagaaattaaaaatgtaacgaccatataatccagtaaccccattcctgggtgtgtatccaaaggaaatgaattgagtatgttgaagggctaacaatactcctacatttatgggaatgctattcataatagtcaagaaatggacacaaactacatctttgtgtggggatgagtaaagaaagcatgacacctgcacatagtgggatgctttgcagccataaaagaatgaactcctgctattcataacagcatagatgcaactggcgggtgttctgttaagtggaataagtctgacatacaaagacgagtacctatgatctcactcatgtgtgggagtgtatgagctgatctcatggaagctaggaggagaagggaggctgcctgaggcttaagcatgcgagaaggggacgaaggaggaagactaagttatcacttaatgggaaaatttagacatgctactgcacagtacagtgactgcagagagtgatgatatactacgttattttgataactctgaagaaagaaatgagaaaagggatatgcatatcctcatttgaatatcatacaatgtatatgtatataaaaacacacattatacccccaagacatgtgctatttttgtgatttgtgtgtcagtaaactatgagtttaaattagaaggctaccatgctatttagtaacaatagtttgacattaagcctttaaaagtcaataatattgataacaacgataatggtatttactctcatattaaatttcttactgctagggctgacacaggagaaacactaaacatgactgatactcaagaccagaataattcttctggtactaaaaatgatttgtggcactatataataaatatccaagcttgctttgtttgatatgggcttcattgtctcaacaggcttggttgtctcctaagacattgttataaaggtattagctggcagcacatgcctgactatctcagtatagtttcaaaacattttgaaagttcagttgctatggagaagttcaaatactctttggattctaggctacaaccaggcttttttattccaaaggtctatgccttcatgttgactatattagttcatttcttccactaatcctaacatggagtcttggcccctaaagaagtccaatcaacaactgctggcttgagttgagtagtcttcagcaattgggtcactattttcttgtttgtgtttagaatataaagaaagatgatcaggatgcttagaggtgtctggcatatgtgaggccttcgtattgccttacatcactgtagggaaaagtccatagtaccaaaacctgaagcataaggacaaaaaatcctacatataattcagtctgtcgttaacattttatgcatctttttcttcattaagatcaattaataccatctaagtgatttatatattttatttttcacttattattttattatacccatacaatttctttgaatataagttatgtgtctacaaaatttccattctacaaacatttcattggttgatcattttgatggctttttcctctttgattatataaaaatcgttttcaatgtaattgtttgacataacttttgataatctcctaggttaattctctagaggcagtgctgtggagacagatgagcaacgtgcaaaagcctacttctctgttcaaaagttgtaaatgtatatttcagcaggaagaatttctattatatatacgacagggattcaatattctttttttctcccccagggctgggatgcagaaaggatatttcaagaggcagagaaattctttgtttctgttggccttcctcatatgactcaaggattctgggcaaactctatgctgactgagccagcagatggccggaaagttgtctgccaccccacagcttgggacctagggaagggcgatttcaggtagttgggttgtcacttacagtgcagctactaagtgagaaacagtgtatgaagggttcattatacttctttgagcatgtgatttaaggatctatgtactaagactggaagtccaagggagaacatttcttgcacccaggaaaagaagatggtccacagttccctccctgcacttatagagaacatagcttacttgaaaattttatgctcattagagtctcgtggtcacagggttcattggaccccctgttaggaaaggtttggtgctgagaactatgacagttgaccatatgtctcacacgctgtctctcactcttgtgagacaggacaaatgaataacaaattggaattaaaaggagaaaatagtacattctataagagagaattgagagagcagaatagaccgctcctggactaggagatagggtcagacaggtgctttgtgattggcttaggaattgcacctgaattgcaagaagaggttaggcagactcctgggagcacagcaggttggaagaacagaagtatcaggacctggtgtgggtgctgacgccggcaagtttagactagaacaacaatgacaaaaaatatgtgtaggacagtgtgagtctgctatgtggcagaatgagcataggctttggaatccaaggtgtgtcactgaatgatcaggcaaccttgagcaaagtaatcttgggttccagtttccttatttataaagcagaactctaatgcccaggttgcaagtttgtgtcttgggtttgctccctggcttcagctggtgattttatgtaaggggagaccctctcagacaaaggcaagtgagggaacagctctggacaggcaaagcagataagaatgcaacctgcgatagagatttggctccagtctgatctcatgaaccactccaatgccagatttaaatcaggattggtcacaccttgtggcaaggggctatttatatctatcctgtattagtcacccactggctgtagaatatatatatatatatatatatatatatatatatatataccctccttgcactggagaggtggttcagtggttaagagcaccggctgctctttgagaggacaaaagaatgtttattttaatcacacttttattagctcatctttggcctaatacattgaaattagtcctggcagatcaggaaacgcctcgattctcttgtctgggcatttgcatccatgttccctttccatgcaaatttgatggtgtgacattctaaagccgaccactgatgagaggtactttcagtgtctatgtgcagcagggccagagtatctgcccagttcaagtcatttcatggtagacatcatcttaagaatttagcttttcgtctcttgtcacttacacactactcatctcagaattatgtagcatagagctctatgtatcattttctgttagtgtaagagcacaagaatggaacttagtcctttatattgagtaaatggttcataaggggcaaatagtaaaacctcagtgatgaatagatcttcatgtcatccgacccccacaaaactctactctagcaatgaaaggaaattaagtaagattcactttaatcttgtccgtttttatgcagaatcaagatgtgtacaaaggtcacaatggacaacttcttgacagcccatcacgagatgggacacatccaatatgacatggcatatgccaggcaacctttcctgctaagaaacggagccaatgaagggttccatgaagctgttggagaaatcatgtcactttctgcagctacccccaagcatctgaaatccattggtcttctgccatccgattttcaagaagatagcggtaatttttcttttggcttgtttgggggctaatctgatatagggaaatgtatttaacagcatcttataggaaaatacttgttttactttatgctacttggggggaaaatgtttatgggacttgctactgatataaagcttctcttttgtaaagtgtggatccgagcatgcaaactgtggtttaaacaaggaattaagtgagataactgaggaatggcctgcaaatgatctactagaagctggatgggatttgggaagatgataactagcccacagaacttaactggtttgttgaagccatggagctgctcagtgtctggattttaaatagttggctgtcatttttaggttaaaaacaaaggacttccccagaatgttagagtaggaacctggcattctgaaactatttttacagagaaggcatgtgaacaaaagacccattccatccctcaacaccagtagctgaggcagcccatctcttttttttgggggggggggcttgaggaaacactacaacttttcaagaggcccagattatccagtgaggttgtcatggcttttgcttctttcatgaatttatcacccctcttttcactatttttttttaggttctgtaaatttttttttttttacttttcataatatatgtatatatgcgtgtaggctgtgcactcattcctagcctccatcattttctttcttattcctgtcccctatctctaaaaccatgctttccaacaaatctccctcctatttcatgtctttttgtttgtttgtagcccactgcatttaattagcatttgatccaagccttggttctcctgtaagaaacacctagtcagtgaagtcaaggtcaggagttccatccttgtgtgtacaattctttgtcttggtctctggcttttagtgatctgacaaaggcctggtagcaggccatcattatgtctgatatctggttagtcactattaaccagatatagtagtttataaaccagtagtggtgctttttgatgaagaaagcaacatgggaaaggcagtttatggtaaccatacttccttcataattttaaaatataattcgttcatactaatgaacagataatgatttgttttatgtttctttttttacatctgccttgagaataggcaaagatgatgagatcggtatgttttttatgatgtcacacaacgaaagcattagcttgaaacattccttctttgcctctggccttgatttgtgagtgatgttaaaccaaacagaatcctctgtagccgtggatgtcctggaactcacaacagtctttctaccacagcctcacaagttttgggattttaggcatgagtcaaagtgcctgcctctttatttttctaaacaaatttactgagcacctaccatgtgtttggcatttttcctagacacacagagtagataataatccctgcattcacattttagcttctgtgatgtgctggaggcagccctacttcctctttaggctaaagaacatacacaacttgtaaaaacatttaatgttcttaataagtacggctacagagtaaaaccctgtcttcaaacaacaatgcatacaaacttcaccagagaaaagaagggcaagaggaagggaaggaggagaaaaatgaaggatcagtccatttgaagtccattgtacttttgagtatccagtggatggtatcctatagagatgtccatttagggtgtttgtgtgtgtgtctttaaatttattttcagctaatatatggaaatacaatgggtgcatatgttaatggagtagcatgacactatgaaacacgtgtaaatcatgtgctgtttctattacagcaaatgcatctacctctacaagcaccatttaccagtagtgaaactttggagggttctttcttctggcttctttttcttttttcttttcagtgctggagacaggactgaggctctcattcgcgttaggctctgtcactgtcactctgtcactgatagacatctctctgaaatgcacgagacattgttgtcatttacatagccagtctattgtgttaaaaaaaaatacctccctgccctggtttacaaggtttcaccacatctgtttaatgtttgcttatttactaaggactcacaaaagagcattacagagcagacactggactgggtatacagtcttgaataagacattaacagacactggctacatgcagctctcaagaataaacagtatgcttgggaaagtagatgaaattgccctaattaactctgataagggggtatttaaagaaggcagttgaaactagtaaaaagtaaggattagttaatttgtggattttacttacttgttctattccagggctttagcctgttccaaatccatcatccgagaagataaacatacaacacacacaatgtccactgccaatatttctcatgtaattatcctgcagtaactccaccttgaggaggaataatagtttgtgattaggaaagttctcttacaaaacgtatgcttctgtttccatcttaatatttgttcttttccgttttactttcagaaacagagataaacttcctactgaaacaggcattgacaattgttggaacactaccgtttacttacatgttagagaagtggaggtggatggtctttcggggtgaaattcccaaagagcagtggatgaaaaagtggtgggagatgaagtaagtcaacgaatttgaaatcagcaaaatgtttcctcatgggtttactgtttaattaaaagcatgttgctaaaccacacacacacacacacacacacacacacacacacacacacacacacacacactggtatttctatttcattgcctgagggcaggctttttagaggtctgaactcaaatgccaggaggggctaacacactaatgtcatccctatcagggcctcaatcaatgacagatgggccggttggtggataaataccttagctcctctgtatccctgagtcacatccacggagtcccagacttccggggaaagggcatcatttcacattacttatttactgatatgcttgataacatgtcctttgggaggagtcctgctttcctttctcagcactttgatcatgtttcctgaaatcatttcctagaatattgtctcaaaagtcattttccaaaaggtaagacaagaaatgtgtaattaattagcagtgtttctaatgtttgtgtataagcaaagctgtccacaaagagctaaagttgcctacaaaaatctcaacaataaaggacaaaactatattccttttacctaaagtccaaaactaacgcctatatggcttttctaccattttttctgactgggataacctttggatccctgctaatttattttaattattatggaatcagacccttaaagtatcccatcaatgtatggattgctcatttggtcatttatcaatcagaagtgaagtttctgcttagtattctggcacagatctattactagacctctaaagatgactaagacacagttgtatcctctcagagctcatgtctcagtggcaagggggagtttacatgcaaaaagccacacatcacagggaaagacatggtaaagaattaaggaaaacagtggagataatacacaaaagtaggaggcctcagaggtataggaaggggacagtgtgggggggggggtggcaggatatgataataaggacttgaagatgatcaaaatatattagatacaggtatgatgtcacaagaaccatgattttatgtaactaataaatactaataagtaaagtgcaagggaataaagcattactaaaagaagacctagatgctaagggatcttaagggattaactgccttagaaggtgggtggcaggcttcacatcttaattagctttgaagaataagatttcaattttacaaaattctgtccaacaggagcacataacatgtgatgtctgagaggaatttataggtttttcacagcatatttgtaaccatacaatgtgttttatgcacattcttagtgttttagaatatagatatgttctaatggtgtgtgttgattgaattaaggcaatgtgaggaaagctcacagcacactttgttttctggcttttacaatgattctattttattttaactgacaatcagtttcaatataaatactattgccaggtgagactgctgtagttgagctcagatctgccttctcttgatggtccgtccctcactaatgtagacttcccttttcttcccaaggcgggagatcgttggtgtggtggagcctctgcctcatgatgaaacatactgtgaccctgcatctctgttccatgtttctaatgattactcattcattcggtaaatgacagttttctcctctctgttagtaaatagggcttaaaggatagctggggctaatgacaaaaacttaaacagtgctagatgaagtgataattacaaggcttgtctatctccttcctatccccaagcttaaggtagattatcagtttcaattcaatgttttatcaggttaaataaacttatacgaggtcttttgcctattgttaaggattgtcctactttgctttgtatagctgtgatagatactatgatcaaaagcaacttggagaggatatggctttgtctcttcattgaaggaagccaagacaggaactcaagcagggtaggaggcaggaactgaagcagaggctgctgaggaacactgcctactctcttgctcttcatggctggcttagcttatcttcttgtacctcaaatgatccacctgctcagggggtagtaccttgtccagtggtctgggccctcccacacaaaacatttagccagaaaatactccacagactagcttagaggtcaatgtgaacaaagcattttctaaattgaggttccttcttcctagatgactccagcttatggcaagttgacaacacacacacacacacacacacacacacacacacacacacacacacgagtttattatgcacttaaggagtgtttttattattcttgtttactttgtctgagacagagacatattatatgtcccaggctggtctcaaactcctggatgcaggcacgtgtggacctcacctcctgtatctgtaaaaatgggaatgtatttacctggcattaggaccagcttgataccactttataggaaggagctataaataactctggcacagcaatccaaatgtgactaacctgtgacacagtagcaaaactgttctacggccaaatctatgcaatgcaaagagtatgtgcggcatctgaagagttcatggcggttttcttctagagaaagatttttcacctctgggattatttgggccaggaatttaattatttccctggatctgtgactctactacaacaataaaacatgagccttctttttgcttgtttctttgttacttgtacagatattacacaaggaccatttaccaattccagtttcaagaagctctttgtcaagcagctaagtataatggttctctgcacaaatgtgacatctcaaattccactgaagctgggcagaagttgctgtaagaaatgcctcaatgtgctgaacctctcttagcatcccatatgactaatttctatgcctatgatttgatttaatttatttgcttcctccgcccccaccccctcccaaaattggaacttcagactttctccaatgaaccatgaaatgaatgttcaatttggaatgagacaagaatctaggtcatgtgaagcctggatgggccacctcctgtctctatgtgataactacagggctctcttttgaacaattggaaagaaagagaggttgataatatgccttattttaataatctggcagaagaaaatttagttaattccaacagaagaagtataaacagtgtagatgcagaggtatgcccacctggcacccttcaaaatgactcacacttggttgggttttgtttatttgtttatttgtttcatatagcaagatgctgagtcttggaaattcagagccctggaccaaagccttggaaaatgtggtaggagcaaggaatatggatgtaaaaccactgctcaattacttccaaccgttgtttgactggctgaaagagcagaacagaaattcttttgtggggtggaacactgaatggagcccatgtgagttcactgactgcacacacattgtgcattgttccttgtttgtttctcttggtgtcctttagccatagctttgtgggggctgtggcgtagctcaatatgtgttcctctttcttagatggtgtcacaaactctgtcacatgttcttacttgtctcaccttcacagtaagagtctagtactctcctactccctttgaacctgattctttgccctagattgtttctgttgctgtgataaagataccatgaccaaaagcaacttggagaggaaccggtttctttaagcttatacattacagttcatcatcaggaactcaaggcagtaatctggaggctggaagtgaagaggggccgtggagggacactctagctggcttgctctccaagctttgctcagcttgatttttcccaggaccccctgcccaggggcacttttgcccacagtgtgctgggacttcacacatcaatgattagtgaagaaaatattccacaggccaatcagatggagacatttttcaattaaggttcatcttcccagctaacaaaatgtcaagttgaccaaaacaaaacaaaacaatagcaacaacaacacctaactaaagtactcttgtaagacaaacactgtcatcagctagaccatgtgtttctgacagtcactttaataactttccaaccttgtttcttttgctgtcttattatcaatgttagaactcctctcccagctcctcttccatgtccactgttcactgtctcagtaaagtattgatgggatatttttttataaagcagttatcaatatttatatctggccacctgcacagccaagtaacacagtatgctgcatcctctcttaagtggcttgtctgtatcagagtttctactcacccatttttggcatacacaattcatcatcaagcagaacatttctttattgccaactagtagaatgtatctatttattttcatcccttgcaatttccccttggagttgtacatgttcctctcttaacataggccataactgtccttcgaaatcacctcctggttcttctgagttgaatcccgtttcctggattccatatcttctttccaggtttacttcttgttctattgaagaacaagttccagttggctgtctgagtaagtgatgatggtgtgcagaaccatggcatctccaaattgttttaaccacatcctcagtaccaatttcccatactcttcccacctgagctgcatcagaatcccaagctttctctgggattcactcctttgtggcctgtaatctcatctatgtacacatcaggcagtgacttatgattctctcagaaagcagctaccagcttctcatcatttctcattttgtaaaagctgtgtctgtgccctttataaaaatcctattgccattaccttactgcagtcttgacaagggacaaaaacatgtgtggtcagtctgtcatctaaacccccggtctgaactttctctttaaccagatgccgaccaaagcattaaagtgaggataagcctaaaatcagctcttggagctaatgcagtgagtattgttctaaatggtgattccatttttcctcagttcattctcttcccttgaaatcccattctagtgtggtctaaagcaaaaaaggacacatggtttgcttaaaactaagcccagctctggccttcaactgactccttcctttctcccattttccatttttccctccctccctccctccctccctccctccctccttccctccctccttaacttttttttccctttgtaatccatctcttacgggataggcaatgtctactgtttacttgttaccttgggtgtgttatgaagatctgtgctgaaacatctttactataattatcaaactcccaccttcacaattgactcatctctatgacttgtatttaaggacatggtcatggctatatgatgaggagttgtttcttttcttaggtttttggttgattttgttttattaaccatttatgtacttgttttattaactgtctggtttatggggaaaaatttcaaatctggaagcttgttttaaaagtgcagcatctcaggtcccagccctggaactgcttaatcaggatccccatttttgtcagccctccatgtggtttgcatacattgggatgactgggaaactccgagtctcaagcttggctcaacattggaactgcatgggaagtcttcaaaatatgagtgtgtgggctacacttccagagacgctggcttaattgttctggagcgtgacaccaagatttaagagctttcttttttgtttgagatagggtctcactgtgcagttaaggctggctatgtagatgagattggccttaacctcccagaaagcctcctgagtgctcagattaaaggcctgcaccaccatacccagcctcattaagaggtttttaacctggagctggggtgatgacttggcagttaagtgcacttgtggctttagcagaggacccaggttcgctgtactgccatggtggctcgcaactattcctaagcccagttctatgggttcttgtaccctattttgatctccacgggcaccaggcatgtgcatggtaccagcaatagactcctacacaaaaataagggtatttttaaaatataaggagttttaaattttccccagagattctaagtacaggcatgtttgagaaccgttgacttaaagatacacttttgaataacaaaatatttaatagaaaatcttattatttatagatttataaacatctatctcacttgatccagccctgggaagtacaagtttttaagagagacaattttcttagtatgtagtaataattatacaaagtgggtttcattgggagattttcacacatttacataatttactttgatggtaatgtatgccttcatttattgtctttctcagcccaccttcacttttccccttcctcttcccaagtagtctccttttcaatagcagctttgattaggaaagaggagagggcatgttgtctcaggtagatgggatgaacagcttgggtgtgtcctagaaacctgggacaatcagataaagcagtccaatgttctacagaaaactgacgtgtcaaggtatagactcgatgattcagtttctacctccccatttcctgaaaagttaaatatttcccaagacttgaaaaattttgctatatttttatagatacctaagacaccaatgtacataacacaagcattaaaaatgaaaacacaggaaagcaactttaccctttattgaaaattgattcttttctcatagaatacattctgattacagtttcttctcccagctcctccccagctcctctccactgggattcactccctttctgtctctcagaaaagaacaggctttcagacagcaattggagaagcacaagtccattgtttctcaaccctgagtgcaaattcccactttttaatgtgcattgaaagctcaaaagaatatttattttgatcacacttttattagttcatctttggcctaatacattgaaattagtcctggcagatcaggaaatgtctcgattctcttgtctgggcatttgcatccatgttccctttccatgcaaatttgatggtgtgacattctaaagccgatcactgatgagaggtactttcagtgtctatgtgcagcagggccagagtatttgcccagttcaagtcatttcatggtagacatcataagaatttagcttttcaatgtttaaaatatggaagtcctagccagagaattactagggacaacagttatatacaactatcactattaaaaaacccatacttcacatatgggtaaacccaagcttaagtgaaaacaattctttcctaagattctgtgttgctttttctctgaaagggaccggagaaaggaaactcctttgtcatttattgatctctactgccacccactggaagaaagggcgaatgctacctttaaccctctctggcaatagaatatgcatggttcaaatacccgagacttcccaataaaacagtaaaagctagagttgcctacaaaatacatacagtccacacaaaccacacaactttgggctgtctcctatctataaacctatcctcagggctcttatttcaagtcatatcaaaccaaagtgaaaggaccgatgggcccctgaaacaggaatggttgcattagcacctcccggggtagtagatatgtaaattcttggttctcatcctagattttcgaaatcggaaactcttcatgtaatagtcagttaaatccattccaaccaccaaatgcggatgcatgctcacattggagggcaaagacttgatggccaaaacccttggaatagtgaggaggactctcccatttccctctgcccccgccccattgaatggtgtctttcttctgaatttgcagtatgaatggaccaacaacgaaatgttcctgttccgatcatctgttgcatatgccatgagaaagtatttttcaataatcaaaaaccagacagttccttttctgtgagttgatttgctagattttattattattattattattattattattattattattattattatttgctttacatcccattatcagttccctctccttcaagtatcccctcacccaaatcctcccctatttcccctccccttctcctttgagaaggaggagcacccctgggtattgccctgcccctggctcatccagtcattgcaggactaggcaatttgctagattctgtacataataactccctgctccactactctagagcttaaactttcttcacatcaaatcaaggagaatatccaaatatactttagtttctgtatttttgaatggagatttcacatatgtatatgctgcgttttgaagttctctcatacgattcccactgaattctttcttctactcttactcttgtacctctttttctattgttaccttgtttatttggagtctactgatcttaactagggttgctggcataagcatagtccagaattttatttggcaaccaggaatttgggttccttttcttttaagatgcttatggtttatcttgtagatgctatgaacgttgttattaaacatctctctctgtctctgtccactctcatctctctcatctcactcttagaagtaaagtagaaaaaaaaagaaaattcttttcttcttggagcttatatcaggagagagagagagagagagagagagagagagagagagagagagagagagctatggaggaaaacacacaataggacagggagaggtaagaggctgggggaatgttcaactttaagtgagttagcgattagaaaggacaccactaacattaagaagtagggagagaagcagaaaagccaacagtgcagacaatgggagaaattgttcagcaagggaaacaacaggcacaaagtctaacgaagtaagagccattgcatggcaatggaacagcaaggccattgtggatggggaagcatctactagagggcaaataggtgatatgatcagggcagttttagggcaaaaatgagcacagaaagttcattggggcctcaaggccattgcatagactttggtttttggggtggtcaagtagttattcaaagaccatagatctccacatggtctgccttgttttttgctaggatcactccgggagctgtgtgagcattgctttgtcacactgtatagtgagccctacttttaccttcatcaatagatgctatacaacacttgctggatagagccaagccagagaatgttaacattctgtctctgacactgaagcatcactgtctaccatgttgcttctcatacttataggtcaccaattacttgtcttgattctctgtgtgtctccgtctctctctgtctctgtctctgcctctctctctctctctctctctctccctcttgagacagggtttctctgtgtattgctggttattctgtggaccaggctggccttgaactcatagagatctgtctgcctctgcctcctgagtactgagattaaagcatgcaccaccactgcctctatagaattcttgattcccttggtgttttaggttgacattaaaaagaattgaacagatgtaggttttgtgtgatagtcaggaaaaacagtatcgaaggtggtcttacaaccctgcctctttctctgttttcaagacccggtttctctgtgtaacagccctggatgtcctggagcttgatttgtagactacgctggtctcaaactcaaatatctgcttgcctttgcctcccaagtgctgggattagaagcatgcattaccatgcttagaatgtggggtattacaacttttatgctactgatttcatgtgctttggatagtgtcaagattcttggtcaattctatctttctactgtttgccctgttctgtgtctcctcatccaggagccatgttctaatctccattccactggcagggggaggactttcttttttctttctttctttctttctttctttctttctttctttctttctttctttcattaggaaagaagaaatatatgtttctttttgcccccccccacagagaggaagatgtacgagtgagtgatttgaaaccaagagtctccttctacttctttgtcacctcaccccaaaatgtgtctgatgtcattcctagaagtgaagttgaagatgccatcaggtgacattctactttcaaacaaactatgtaaagatggagttagctaatgcgccaatagcttacatttcgtgtaatattgtgtgctggacatcatttaaaatcatttatgccttttacctcatctcaaaacatgggttgttcttgtggctgtggtggtggtgtctgtggtctatgtatggatttgtgtgtgggtgtgtacatgtgacatggttacacatgtagaagccaacggttgacatagctgtcttttctgacccatcaccaccttgcttttagagacatgatgtctcactaaacccaatgttaatcaatttaggtgggctggcaaatgaacttcagggatctgcccatctcccccaattcctagcatcggagtagacacatgctgggtactaccacacccagctcttttatatgtgtactgggcatcagaactcaagtccttgtgcttgtacagcaggcattttgctggtggaaccatctctttaaccacacaaacttgggttcttgatcctattataatccaacagcaagagactggttttgattggggactggccaggagaaggtcttgatttgaaagcaagcaagcaagcataagcagtgctgagaagctcctctagaaactgtggacaggagacagagacctggggaaatgtgaccaggttccaagttatcaaacaagctatcattgtcaggaactagagtttctgctaggcagtcccatctgacaactctaggatacggtagagtgtgtacccgaattgtctcagcagaggactgcaggagctcagatctttaacacttctccctagtcactggccaggaactctttctggtgatattcattccctagtctctctagcctgacaatggtctcaggtaaaagggaagtcattttggctaaaggaagtttggctatcatgcttggaaagtgaagacaagaaatgggcacacactgccaccatttgtacagggacattatgacattatgaaagagttattgaattgcccaaagcaggaatcaaggaaggtaaacattaggttcttttcacaccaagataagaatttaccatggagcagtgatcactcgtaagaaatcacttgtccagactgcctcagttgttttaaacaagatagcaggaatggaggacagacggttctttttcttattaaaataaaagaaagagagagaggaagggagggagggagggagggagggagggaggtaagtaggtaggaaggaaggtcacaagcaagcaaaatgcttactaagtttcctgtccctgacacctcactaggtggcactattgagctatgccaggaattctatttacccttcagaccagatctcagacctgctgaatagggtgtgcctagaaaatttggcctgattgaggtgtgtgaatcatgcccaaagattcactctcctcctccaaaacggctagtatcagcctcagacaaaatgctgcctgagattgtcatgaaagcaccttctctgcctggattccgagatagaagctacaggaaatctcaaactttcttttctagaaatgactcttaaaaatgacttgattaactcttgaggttgccagggcttatggctacctctgtgttggctgcttgtcctctctgatggaacctgaccttgtcacctgagctataccaatggatgagaagctgtgggctagacattttactctatgaaccaaaatgtttctccattatactgtttttgttgtcaacaatctccatcctttctactccgtcaacttcagctacttataatttcagtgcttgagaaatgcagaggtctcatttccacgccccactgtgacgctgcttccaaatgtcaacttcttcagcgacttgggtgtaggatatgattcatttataaaagagggattttcaagtatgcttttctgttttttgatgcaatgtcattttaataaaataattgattgtttgagattattttgagacaaagcttctctatgtagcctcggatgtcctggaactcattctgtagacaaggaaggtctcaaactcacagagatccatctgtctctgcctccccagtattaggattaaaggcatgtgccactactgcttggttgtcagtacttattttagatgtaggctgaagactgagaaattcttgccgaatatgctaactttacttttgaaatgtagaatctatacatgataaatactaagctatctgttcattctaaaatcttctttttataatgaaacctatgaatttgtgtgataactttattctcttaacgttttagtgtgtgagcctatgtgtttatatgtgaactgcatgtgtgcagcacctgtggagtcaagaagagggctttcaatcccttggggctgaagttataggcacttttgtgagctgcccaaagtgggttctggggaccaaactcaggtcctctggaagagtgatacaaaacctcttaaccactgagccatctctctaggacacttctctcccccacccctctttataaatttctaacttaaacttcaaataaggttgttgatagacctaattcaggttaccaggtatgaaagacatgtagttgtcaaaagtttgtatctctctattggccctatcttcttcaattttatctttattttaggtgtgtatttttgtggctttaaaaaagactgccttgctatgttcccctgatgatactggatcgtgctttgcagagttgattgactcaaatttgtctaatcctcatactaaagcacggcttcccaggcatgtgccactgcacaaagttttagttgtacattctaatgacaaagcatattaaatccctttgggaaccctgatataaggagaaaataatggtgcagaattttatttggtctaagcatcccatttactgcattctcttttgcaaataggatgtctcggggccgcatcaatgatgtctttggcctgaatgataacagcctggagtttctggggattcacccaacacttgagccaccttaccagcctcctgtcaccatatggctgattatttttggtgttgtgatggcactggtagtggttggcatcatcatcctgattgtcactgggatcaaaggtcgaaagaagtgagtatcctttcctagatttatctgtctttttagaatgcatgaattggaaattttgcaacaaataacactgtatccacacaggataattacttacttggacatttatctccctaaatattctgatatatcccatagaatcttgtcagatgctagcagtaccttaccttactagcaagctcacatattatagatgtctagcctgaggtttattgggatgctgtagtcaagtgcatcctatccacagtggcatagccagactgcaaactcagtaaggcattgttctcactcatgactcttgcttcatccacagagactgacatggagcacaaagtgtaatgtcatctaagaatgaggtgcttcaaactactactctttagttactttcattcctccttgggaaactcaaatttatcatttgagttcatgggcaggcaccctttaatttgttctagagctgagttgattgagggctttaatcaccagggactgacttctagctcctgtcttgagaaagtattaactcttgttggtctgttcaaataacttagtattttgtatcactctgacaacattatctgcatgttttcagttgtttaaacatactctcagttgaatctttacacaaattagtttattttatttaattttctttactatagagatctctcgattctaccaaataacttcatttgaaaaagcaaggctgtggagatgggtaaaatcacctgccccacaagcatgaggacctgactttgaatccccagagcccatatataaagcagatacaggaggcaagcatctataatcccagcactcctccagggagatggaaggcagaggcaggaaagtccccagaagtctgtaggccactcagcctggtgtacacagcagaggcaacagaccccatctcaatcacaatgggtgttgagaatcaacactggatattgctgtctgatccccacgtgtgcaatgtagcatacacacttacacacatgcacatgtgtacgcatacacagatgtgcacaaagcactttctaggtagtgcagtcaacatggttacaatgttcaaaaatacaagggggtcatagagttccgttaccattctttgtttctgagtttccttttaaccaaagttatcagtttttagtaaaatgaactatgcactacatattcttatttaccaactagtatgttttagaggtatttccagatttttcaaaaaattcccaagcaatatattttagagattctttcagattggctatgtaaagcacttccatttgcctggtaatgtagttgcccactttgtagcaaatgggtactcccatggttaccaaatgtttgtgtctatttaaaaaatacaacaatttcaaataggcaatttcctgtgcatgctaaagtaggtgcaagatatgtcccagcaggtaaaaggctaggttaaaatcatcatgaattctaattttaattaatacttctgaactgttctcataggggcagaattaatgtacaaacagcaccagcttatgaaagctctccttttctccaactcagcatccttagcacagggtgccatcagactttttgacagttatcctttgggtcagtgtaagtaagtgtctttctaccatcactttaataaggagactttgttttatttgacaggaaaaatgaaacaaaaagagaagagaacccttatgactcgatggacattggaaaaggagaaagcaatgcaggattccaaaacagtgatgatgctcagacttccttttagcaaagcacttgtcatcttcctgtatgtaaatgctaacttcatagtacacaaaatatgagagtatacacatgtcattagctatcaaaactatgatctgttcagtaaacgttgtccaaagagcatcagacttgagtggacatcttcactgacattgctttcagtatttatttctgcctaaggatttgacatctcttctgtttattaatagagatgtttatcttagcataaaagagggaaatgtgcctttggcctcacagtctatccagggtgatatggttgggtaactggagttagaagatgagatgatgtctcttgggggcaagtgttggcttcggtgtggcatctgggctgtgaactggtgggactgttgaggttgagaatggtgctcgctggtcacttgaatccaagtgtgacgtcatgctctgtggcttctgccttcacacttctcacttcaagtactgtaggaatttgttcacagtaatacttgaaatggactgtcccttctttggaggtgcagttcaacggagaaagaagccagacatcaggtagagaccatgaccttttctcttccaaacttgatcaacatctctctaacaagacacagctagcacaggaaactccacgaacccagagcatgcctgtcagaaactacttccattattctcccattgtggagtaagggaaaattccagatgaatgctcgatctgtgagatgggtgcccagtctctgaaattgtttgtattttctcacagggtctgagcaatggtgaacacaaagccgacctcaataaatacttattagatttagacactcctct

SEQ ID NO: 52 - DNA, 2418 bp, Mus musculus
  source 1..2418: mol_type = other DNA
atgtccagctcctcctggctccttctcagccttgttgctgttactactgctcagtccctcaccgaagaacaggctaagacatttttggacaagttcaaccacgaagctgaagacctgtcttatcagagttcacttgcttcttggaattataatactaacattactgaagaaaatgcccaaaagatgagtgaggctgcagccaaatggtctgccttttatgaagaacagtctaagcttgcccagatgtatccactacaagaaatccagactccgatcatcaagcgtcaactacaggcccttcagcaaagtgggtcttcagcactctcagcagacaagaacaaacagttgaacacaattctgaacaccatgagcaccatttacagtactggaaaagtttgcaacccaaagaacccacaagaatgcttattacttgagccaggattggatgaaataatggcgacaagcacagactacaactctaggctctgggcatgggagggctggagggctgaggttggcaagcagctgaggccgttgtatgaagagtatgtggtcctgaaaaacgagatggcaagagcaaacaattataacgactatggggattattggagaggggactatgaagcagagggagcagatggctacaactataaccgtaaccagttgattgaagatgtagaacgtaccttcgcagagatcaagccattgtatgagcatcttcatgcctatgtgaggaggaagttgatggatacctacccttcctacatcagccccactggatgcctccctgcccatttgcttggtgatatgtggggtagattttggacaaatctgtaccctttgactgttccctttgcacagaaaccaaacatagatgttactgatgcaatgatgaatcagggctgggatgcagaaaggatatttcaagaggcagagaaattctttgtttctgttggccttcctcatatgactcaaggattctgggcaaactctatgctgactgagccagcagatggccggaaagttgtctgccaccccacagcttgggacctagggaagggcgatttcagaatcaagatgtgtacaaaggtcacaatggacaacttcttgacagcccatcacgagatgggacacatccaatatgacatggcatatgccaggcaacctttcctgctaagaaacggagccaatgaagggttccatgaagctgttggagaaatcatgtcactttctgcagctacccccaagcatctgaaatccattggtcttctgccatccgattttcaagaagatagcgaaacagagataaacttcctactgaaacaggcattgacaattgttggaacactaccgtttacttacatgttagagaagtggaggtggatggtctttcggggtgaaattcccaaagagcagtggatgaaaaagtggtgggagatgaagcgggagatcgttggtgtggtggagcctctgcctcatgatgaaacatactgtgaccctgcatctctgttccatgtttctaatgattactcattcattcgatattacacaaggaccatttaccaattccagtttcaagaagctctttgtcaagcagctaagtataatggttctctgcacaaatgtgacatctcaaattccactgaagctgggcagaagttgctcaagatgctgagtcttggaaattcagagccctggaccaaagccttggaaaatgtggtaggagcaaggaatatggatgtaaaaccactgctcaattacttccaaccgttgtttgactggctgaaagagcagaacagaaattcttttgtggggtggaacactgaatggagcccatatgccgaccaaagcattaaagtgaggataagcctaaaatcagctcttggagctaatgcatatgaatggaccaacaacgaaatgttcctgttccgatcatctgttgcatatgccatgagaaagtatttttcaataatcaaaaaccagacagttccttttctagaggaagatgtacgagtgagtgatttgaaaccaagagtctccttctacttctttgtcacctcaccccaaaatgtgtctgatgtcattcctagaagtgaagttgaagatgccatcaggatgtctcggggccgcatcaatgatgtctttggcctgaatgataacagcctggagtttctggggattcacccaacacttgagccaccttaccagcctcctgtcaccatatggctgattatttttggtgttgtgatggcactggtagtggttggcatcatcatcctgattgtcactgggatcaaaggtcgaaagaagaaaaatgaaacaaaaagagaagagaacccttatgactcgatggacattggaaaaggagaaagcaatgcaggattccaaaacagtgatgatgctcagacttccttttag

(19) United States
(12) Patent Application Publication
(10) Pub. No.: US 2026/0305587 A1
(43) Pub. Date: Oct. 8, 2026

(54) HUMANIZED ACE2-EXPRESSING TRANSGENIC MOUSE SUSCEPTIBLE TO SARS-COV-2 VIRUS INFECTION AND METHOD FOR PRODUCING SAME

(71) Applicant: SEOUL NATIONAL UNIVERSITY R&DB FOUNDATION, Seoul (KR)

(72) Inventors: Nam-Hyuk CHO, Seoul (KR); Uni PARK, Seoul (KR); Kyeongseok JEON, Seoul (KR); Hyeran KIM, Jeollanam-do (KR); Han-Woong LEE, Seoul (KR); Jae Hoon LEE, Seoul (KR); Mi Yeon PARK, Seoul (KR); Sun Ha PARK, Seoul (KR); Hyun-Soo CHO, Seoul (KR)

(21) Appl. No.: 19/483,851

(22) PCT Filed: Nov. 4, 2024
(86) PCT No.: PCT/KR2024/017199

§ 371 Date: Nov. 12, 2025

(30) Foreign Application Priority Data
Nov. 6, 2023 (KR) 1020230151409

(51) Int. Cl. A01K 67/0278; C12N 9/22; C12N 9/48; C12N 15/11; C12N 15/90

(52) U.S. Cl. CPC A01K 67/0278; C12N 9/226; C12N 9/485; C12N 15/11; C12N 15/907; C12Y 304/17023; A01K 2207/15; A01K 2217/052; A01K 2217/072; A01K 2227/105; C12N 2310/20

(57) ABSTRACT

The present invention discloses a humanized ACE2-expressing transgenic mouse, which is susceptible to infection by the SARS-CoV-2 virus, obtained through the application of the CRISPR system, and a method for producing the same. The humanized ACE2-expressing transgenic mouse of the present invention reflects symptom severity and mortality patterns according to age, sex, and viral inoculation dose, as observed in human COVID-19 cases, and reproduces the immunopathological characteristics of SARS-CoV-2 infection in the lungs without viral replication or lesions in the brain. Therefore, it can be usefully employed for understanding the pathogenic mechanisms of SARS-CoV-2 and for the development of vaccines or therapeutics.

### TECHNICAL FIELD

The present invention relates to a humanized ACE2 transgenic mouse susceptible to SARS-CoV-2 viral infection and a method for producing the same.

### BACKGROUND ART

Since the end of 2019, the COVID-19 (coronavirus disease 2019) pandemic has persisted for several years, serving as a reminder to people around the world of the risks posed by infectious diseases. COVID-19 is caused by severe acute respiratory syndrome coronavirus 2 (SARS-CoV-2), which is presumed to have been transmitted from an as-yet-unidentified animal host to humans.

The pandemic has continued despite rapid vaccine development and international efforts, due to the highly transmissible nature of SARS-CoV-2, its continual mutations, and the resulting changes in pathogenicity, transmission modes, and immune evasion. Although significant progress has been made in COVID-19 research to date, many important questions remain unresolved, including the origin of the virus, the factors influencing its transmissibility and pathogenicity, and the underlying mechanisms of its diverse clinical manifestations. Moreover, elucidating the characteristics of rapidly evolving viral variants and developing effective vaccines and therapeutics against coronaviruses with varying pathogenicity remain ongoing challenges.

Animal models are indispensable tools for understanding the pathogenic mechanisms of SARS-CoV-2 and for developing vaccines and therapeutics. Among them, mouse models are the most widely used in biological research due to their rapid reproductive capacity, relatively low cost compared to other animal models, ease of breeding and maintenance, and the feasibility of genetic modification. In humans, the receptor for SARS-CoV-2 is angiotensin-converting enzyme 2 (ACE2). ACE2, a cell membrane-bound enzyme, degrades the pro-inflammatory molecule angiotensin II to maintain the balance between vasoconstriction and vasodilation, thereby ensuring safe blood pressure regulation. It also functions as the entry receptor for coronaviruses such as SARS-CoV-1 and SARS-CoV-2. However, the binding affinity between murine ACE2 and SARS-CoV-2 is extremely low, and thus wild-type mice cannot be infected with SARS-CoV-2 in a way that reproduces COVID-19. For this reason, studies have attempted to generate COVID-19 mouse models by genetically engineering mice to express human ACE2. Nevertheless, existing COVID-19 mouse models have several limitations.

For example, the representative K18-hACE2 mouse model was developed to express human ACE2 in mouse epithelial cells under the K18 promoter. However, ACE2 expression in this model is predominantly localized in neural tissues, leading to frequent central nervous system infections following SARS-CoV-2 exposure, which differs from human pathology and raises concerns about its clinical accuracy. Furthermore, ACE2 expression in the respiratory tract—the primary target of SARS-CoV-2 infection—is limited, restricting the ability of this model to replicate respiratory symptoms such as pneumonia observed in humans. While K18-hACE2 mice exhibit severe pathological responses upon infection and may be useful for studying severe and fatal cases of COVID-19, they are inadequate for modeling mild or asymptomatic infections. Additionally, the immune responses of infected mice may not fully align with those of humans, further limiting their applicability as models.

Other COVID-19 mouse models have been developed and reported in international journals, including HFH4-hACE2 mice that overexpress human ACE2 in pulmonary epithelial cells, AC70 CAG-hACE2 mice that overexpress human ACE2 systemically under the CAG promoter, and humanized ACE2 mice in which the murine Ace2 gene locus is replaced with the human ACE2 gene. However, these models also share similar limitations with K18-hACE2 mice or fail to reproduce severe forms of COVID-19. Furthermore, since these models alter the tissues, cell types, and expression levels in which ACE2 is expressed, there remains the possibility that the in vivo function of ACE2 in mice is compromised.

The present invention discloses a transgenic mouse that expresses a humanized ACE2 in which specific amino acids of murine ACE2 are replaced with those of human ACE2 through genome editing technology. This transgenic mouse is susceptible to SARS-CoV-2 infection and exhibits infection patterns similar to human COVID-19, and thus can be effectively utilized in research for the development of vaccines and therapeutics against human COVID-19.

### DISCLOSURE

### Technical Problem

The object of the present invention is to provide a transgenic mouse expressing humanized ACE2 that is susceptible to SARS-CoV-2 viral infection.

Another object of the present invention is to provide a method for producing the said transgenic mouse.

Other objects or specific objects of the present invention will be set forth hereinafter.

### Technical Solution

As confirmed in the following Examples, the inventors produced an ACE2-humanized mouse model by employing the CRISPR system to substitute four amino acids in exon 1, four amino acids in exon 2, and one amino acid in exon 8 of murine ACE2 with the corresponding human ACE2 amino acids. Upon comparison of this mouse model with wild-type mice, no differences were observed in the tissues expressing ACE2 or in blood pressure, thereby confirming that the process of generating the ACE2-humanized mouse did not induce any artificial alteration in ACE2 expression or enzymatic activity. Furthermore, when infected with mouse-adapted SARS-CoV-2 virus, the model exhibited symptom severity and mortality patterns according to age, sex, and viral inoculum dose, as observed in human COVID-19 cases, and reproduced the immunopathological characteristics of pulmonary SARS-CoV-2 infection without viral replication or lesions in the brain.

Human ACE2, as disclosed in Sequence ID No. 1, consists of 805 amino acids encoded by 18 exons; murine ACE2, as disclosed in Sequence ID No. 2, also consists of 805 amino acids encoded by 18 exons; and the humanized ACE2, as disclosed in Sequence ID No. 3, likewise consists of 805 amino acids encoded by 18 exons.

Based on these experimental results, the present invention is provided. In one aspect, the invention relates to a transgenic mouse expressing humanized ACE2 that is susceptible to SARS-CoV-2 viral infection. In another aspect, the invention pertains to a mouse embryo or pluripotent stem cell having the property of expressing humanized ACE2 comprising the amino acid sequence of Sequence ID No. 3. In yet another aspect, the invention relates to a method of producing a transgenic mouse expressing humanized ACE2 that is susceptible to SARS-CoV-2 viral infection.

Herein, the term “mouse embryo or pluripotent stem cell having the property of expressing humanized ACE2” refers to an embryo or pluripotent stem cell which, when implanted into and engrafted in a surrogate mother's uterus (or oviduct) and developed into an individual, results in an organism that carries, in a heterozygous or homozygous state, a gene encoding humanized ACE2 comprising the amino acid sequence of Sequence ID No. 3, thereby exhibiting the property of expressing humanized ACE2.

The transgenic mouse of the present invention that expresses humanized ACE2 refers to a mouse expressing ACE2 in which four amino acids of murine ACE2 exon 1, four amino acids of exon 2, and one amino acid of exon 8 are substituted with the corresponding amino acids of human ACE2, specifically the humanized ACE2 consisting of the amino acid sequence of Sequence ID No. 3. The four amino acids of exon 1 are N24Q, N30D, N31 K, and Q34H; the four amino acids of exon 2 are T79L, S82M, F83Y, and S84P; and the one amino acid of exon 8 is H353K. In the amino acid notations used in the present invention, the number indicates the amino acid position in the murine ACE2 amino acid sequence of Sequence ID No. 2 or in the humanized ACE2 amino acid sequence of Sequence ID No. 3. The amino acid to the left of the number represents the amino acid of wild-type mouse ACE2, and the amino acid to the right of the number represents the amino acid of humanized ACE2.

The transgenic mouse of the present invention that expresses humanized ACE2 encompasses not only the founder mouse (F0) generated from the first embryo modified with a humanized ACE2-encoding gene, but also transgenic mice expressing humanized ACE2 that are produced through one or more generations of inbreeding or outbreeding from the founder mouse. Such transgenic mice expressing humanized ACE2 carry the humanized ACE2-encoding gene in either a heterozygous or homozygous state. The Examples below demonstrate that even in cases where the transgenic mouse expressing humanized ACE2 is heterozygous, there is no significant difference compared with homozygous transgenic mice expressing humanized ACE2 in terms of the expression levels of humanized ACE2 in tissues such as the lung and kidney.

In the present invention, the term “first embryo” refers to an embryo obtained by mating a wild-type male mouse and a wild-type female mouse, into which components of a gene-editing technology targeting the ACE2-encoding gene are introduced together with donor DNA, thereby editing the ACE2-encoding gene so that the embryo carries a humanized ACE2-encoding gene. Such an embryo carrying the humanized ACE2-encoding gene, when transplanted into a surrogate mother, engrafted, and developed into an individual, possesses the humanized ACE2-encoding gene in a heterozygous or homozygous state and thereby exhibits the property of expressing humanized ACE2. In this specification, the term “first embryo” is used to distinguish it from embryos having the property of expressing humanized ACE2 that are obtained from one or more generations of breeding from a transgenic mouse expressing humanized ACE2.

In the present invention, for transgenic mice produced through mating of a male and female, or of either male or female, transgenic mouse, inbreeding such as backcrossing or sibling mating may be advantageous in order to increase the frequency of occurrence of the desired homozygote expressing humanized ACE2.

In the present invention, the term “mouse” refers to the species Mus musculus of the genus Mus. The transgenic mouse of the invention may be of any strain of the species Mus musculus. For example, in the case of the C57BL strain, it may be C57BL/6N used in the Examples of the invention, or C57BL/6, C57BL/6J, C57BL/A, C57BL/An, C57BL/GrFa, C57BL/KaLwN, C57BL/6ByJ, C57BL/6NJ, C57BL/10, C57BL/10ScSn, C57BL/10Cr, C57BL/01 a, etc. In the case of the BALB/c strain, it may be BALB/cJ, BALB/cNIV, BALB/cByJ, BALB/cJRj, BALB/cJ, etc. In the case of the 129 strain, it may be 129P1, 129P2, 129P3, 129X1, 129S1 (including 12951/SV, 12951/Svlm, etc.), 129S2, 129S4, 129S5, 12959/SvEvH, 129S6 (129/SvEvTac), 129S7, 129S8, 129T1, 129T2, etc. The transgenic mouse of the present invention may also be a strain obtained through outbreeding between these strains.

The transgenic mouse of the present invention that expresses humanized ACE2 comprises, in a heterozygous or homozygous state, any gene encoding the humanized ACE2 consisting of the amino acid sequence of Sequence ID No. 3. Here, “any gene” includes all genes that, due to codon degeneracy, encode the same amino acid sequence of humanized ACE2 but have different nucleotide sequences. Preferably, the gene encoding the humanized ACE2 may be a gene comprising the nucleotide sequence of exon 1 of Sequence ID No. 16, the nucleotide sequence of exon 2 of Sequence ID No. 17, and the nucleotide sequence of exon 8 of Sequence ID No. 25, which was generated by utilizing the CRISPR system as described in the Examples below; more preferably, the gene may be a gene comprising the nucleotide sequence of Sequence ID No. 52 (a gene encoding humanized ACE2 including introns is disclosed in Sequence ID No. 51).

In another aspect, the present invention relates to a mouse embryo having the property of expressing humanized ACE2 consisting of the amino acid sequence of Sequence ID No. 3, specifically a mouse embryo carrying, in a heterozygous or homozygous state, any gene encoding humanized ACE2 consisting of the amino acid sequence of Sequence ID No. 3. Such mouse embryos include one-cell or two-cell stage first embryos modified with a gene encoding the humanized ACE2 consisting of the amino acid sequence of Sequence ID No. 3, as well as embryos obtained through one or more generations of inbreeding or outbreeding from a founder mouse that developed following implantation and engraftment of the first embryo into a surrogate mother. The embryos also include those obtained by in vitro fertilization of sperm isolated from a male transgenic mouse expressing humanized ACE2 with oocytes isolated from a female transgenic mouse expressing humanized ACE2 or from a non-transgenic female mouse, and those obtained by in vitro fertilization of oocytes isolated from a female transgenic mouse expressing humanized ACE2 with sperm isolated from a male transgenic mouse expressing humanized ACE2 or from a non-transgenic male mouse. These embryos contain, in a homozygous or heterozygous state, a gene encoding the humanized ACE2 consisting of the amino acid sequence of Sequence ID No. 3, and may be cryopreserved and later transplanted into a surrogate mother for use in the reproduction of the transgenic mouse.

In another aspect, the present invention relates to a pluripotent stem cell of a mouse having the property of expressing humanized ACE2 consisting of the amino acid sequence of Sequence ID No. 3, specifically, a pluripotent stem cell carrying, in a heterozygous or homozygous state, any gene encoding the humanized ACE2 consisting of the amino acid sequence of Sequence ID No. 3. Such pluripotent stem cells include embryonic stem cells produced using the inner cell mass of an embryo at the blastocyst stage, somatic cell nuclear transfer embryonic stem cells produced by inserting the nucleus of a somatic cell into an oocyte or embryo and replacing the nucleus, and induced pluripotent stem cells produced by introducing four reprogramming factors—Oct4, Sox2, c-Myc, and KIf4—into a somatic cell. Since such pluripotent stem cells, when injected into a blastocyst embryo and transplanted into and engrafted in a surrogate mother, can develop into a complete individual, they, like the embryos described above, may be cryopreserved and later used for the reproduction of the transgenic mouse.

The embryos used to obtain such embryonic stem cells may be obtained through mating between transgenic mice expressing humanized ACE2 or through mating between a transgenic mouse expressing humanized ACE2 and a non-transgenic wild-type mouse. The nuclei of somatic cells used to produce somatic cell nuclear transfer embryonic stem cells, as well as the somatic cells used to produce induced pluripotent stem cells, may be obtained from the transgenic mice of the present invention.

With respect to the production of such pluripotent stem cells and methods of generating mouse individuals from pluripotent stem cells, detailed information is already established in the art, and reference may be made to numerous publications, including [J Vis Exp. 2012; (69): 4003], [Cell Research, 2011, vol. 21, pp. 546-549], [Biology of Reproduction, 2010, 83(1): 238-243], [Int J Dev Biol. 2015; 59(4-6): 179-186], [Stem Cell Rev Rep. 2020 February; 16(1): 3-32], and [Cell. 2006 Aug. 25; 126(4): 663-676].

In another aspect, the present invention relates to cells isolated and obtained from the above-described transgenic mouse expressing humanized ACE2. Such cells may be cryopreserved and advantageously used for conversion into pluripotent stem cells.

In another aspect, the present invention relates to a method for producing a transgenic mouse expressing humanized ACE2 that is susceptible to SARS-CoV-2 viral infection.

The method of producing the transgenic mouse of the present invention may comprise producing by mating between transgenic mice expressing humanized ACE2, or by mating a transgenic mouse expressing humanized ACE2 with a non-transgenic wild-type mouse. Alternatively, the method may comprise injecting a mouse embryo having the property of expressing humanized ACE2 consisting of the amino acid sequence of Sequence ID No. 3, or a mouse pluripotent stem cell having the property of expressing humanized ACE2, into a blastocyst-stage embryo, and then transplanting and engrafting it into a surrogate mother to develop into a mature mouse individual.

Accordingly, the method of producing the transgenic mouse of the present invention may, in one aspect, comprise mating transgenic mice expressing humanized ACE2 to obtain their offspring; in another aspect, comprise mating a transgenic mouse expressing humanized ACE2 with a non-transgenic wild-type mouse to obtain their offspring; in yet another aspect, comprise preparing a pluripotent stem cell having the property of expressing humanized ACE2 and injecting the pluripotent stem cell into a blastocyst embryo, followed by transplanting and engrafting into a surrogate mother to obtain a mature mouse individual; and in a further aspect, comprise preparing an embryo having the property of expressing humanized ACE2 and transplanting and engrafting the embryo into a surrogate mother to obtain a mature mouse individual.

The embryo having the property of expressing humanized ACE2 may be obtained through one or more generations of inbreeding or outbreeding between transgenic mice expressing humanized ACE2, or through one or more generations of inbreeding or outbreeding between a transgenic mouse expressing humanized ACE2 and a non-transgenic wild-type mouse. Alternatively, such an embryo may be obtained by in vitro fertilization of sperm isolated from a male transgenic mouse expressing humanized ACE2 with oocytes isolated from a female transgenic mouse expressing humanized ACE2 or from a non-transgenic female mouse. Further, such an embryo may also be obtained by introducing components of a gene-editing technology targeting the ACE2-encoding gene, together with donor DNA, into an embryo obtained by mating a wild-type male and female mouse, thereby editing the ACE2-encoding gene (this being the “first embryo”). Such embryos carry the gene encoding humanized ACE2 in a heterozygous or homozygous state.

To obtain the first mouse embryo through gene editing, three gene-editing technologies may be employed, specifically: (i) ZFN (zinc finger nuclease) technology, (ii) TALEN (transcription activator-like effector nuclease) technology, and (iii) RGEN (CRISPR/Cas-derived RNA-guided endonuclease) technology.

These gene-editing technologies create double-strand breaks (DSBs) in specific genes present in animal or plant cells. All cells possess two repair systems that efficiently repair such breaks: non-homologous end joining (NHEJ) and homology-directed repair (HDR) based on the principle of homologous recombination. Among these, HDR is a precise DNA repair mechanism that does not involve indels (small insertions and deletions). HDR may be achieved by introducing into the cell, together with the components of the gene-editing technology, donor DNA that contains a sequence identical to that of the cleaved region. Here, the donor DNA may be a targeting vector or a single-stranded oligonucleotide. When the components of the gene-editing technology are introduced together with donor DNA, it is possible to generate a cell line or animal/plant organism in which a portion of the sequence in the target DNA (or target gene) is altered, thereby changing its expression characteristics.

In the method of the present invention, the first mouse embryo can be obtained by employing HDR (homology-directed repair) using a gene-editing technology. To obtain the first mouse embryo, four amino acids of exon 1 of murine ACE2, four amino acids of exon 2, and one amino acid of exon 8 must be substituted with the corresponding human ACE2 amino acids. Therefore, together with the components of the gene-editing technology targeting each exon, donor DNA is introduced into the mouse embryo, specifically:
(i) donor DNA for exon 1 comprising a nucleotide sequence encoding the amino acid sequence at positions 24-34 of ACE2, which includes the four human amino acids Q24, D30, K31, and H34, so that the four murine amino acids N24, N30, N31, and Q34 are substituted with the human amino acids Q24, D30, K31, and H34, respectively,
(ii) donor DNA for exon 2 comprising a nucleotide sequence encoding the amino acid sequence at positions 79-84 of ACE2, which includes the four human amino acids L79, M82, Y83, and P84, so that the four murine amino acids T79, S82, F83, and S84 are substituted with the human amino acids L79, M82, Y83, and P84, respectively, and
(iii) donor DNA for exon 8 comprising a nucleotide sequence encoding the human amino acid K353, so that the murine amino acid H353 is substituted with the human amino acid K353.

Here, the numbers indicate the amino acid positions in the murine ACE2 amino acid sequence of Sequence ID No. 1 or the humanized ACE2 amino acid sequence of Sequence ID No. 3. The amino acid to the left of the number denotes the amino acid of wild-type mouse ACE2, while the amino acid to the right of the number denotes the corresponding amino acid of humanized ACE2.

The three donor DNAs may each be introduced into the embryo either sequentially, one at a time, together with the gene-editing components for the respective exon, or simultaneously. To increase HDR efficiency and thereby improve the production efficiency of transgenic mice, sequential introduction may be preferable. Sequential introduction, as illustrated in the Examples below, may be carried out by introducing into an embryo the gene-editing components and the donor DNA for one exon, obtaining an individual via a surrogate mother, selecting a founder (F0) in which the donor DNA has been inserted, mating the founder with a wild-type mouse to produce offspring (F1), and subsequently mating the offspring with a superovulated female mouse to obtain embryos. The remaining two exons may then each undergo further gene editing through the same process as described above.

The donor DNA for each exon may include, in addition to the nucleotide sequence encoding the amino acid sequence containing the human amino acids, additional nucleotide sequences of appropriate length on both sides to facilitate efficient HDR. Such additional nucleotide sequences may comprise two or more, three or more, or five or more nucleotides.

In addition, the donor DNA for each exon may, in order to increase HDR efficiency and prevent re-cleavage by Cas enzymes, include chemical modifications at one or more of the terminal nucleotides, preferably one to three nucleotides, such that the sugar moiety is chemically modified, or the base is chemically modified, or the phosphodiester bond between nucleotides is chemically modified. Examples of sugar modifications include substitution of 2′-deoxy with 2′-F, 2′-NH2, or 2′-OMe. Examples of base modifications by chemical derivatization include modifications at the 5-position of pyrimidines, the 8-position of purines, the 4- or 5-position of uracil, and modification of the exocyclic amine of cytosine. Modifications of the phosphodiester bond may include replacement of an oxygen atom of the phosphate group with sulfur, NH2, or other substituents. With respect to such nucleotide modifications, detailed descriptions are well established in the art, and reference may be made to numerous publications, including [Cell Reports. 2016; 14: 2263-2272], [Nucleic Acids Res. 30(24):138, 2002], U.S. Pat. Nos. 5,719,273, 5,660,985, International Patent Publication WO 2011/130195, U.S. Pat. Nos. 5,660,985, 5,756,703, 5,580,737, and others.

The components of the gene-editing technology, in the case of ZFN (zinc finger nuclease) technology, comprise (i) three to six zinc finger proteins arranged in tandem and linked to a Fokl nuclease domain, and (ii) three to six zinc finger proteins arranged in tandem and linked to a Fokl nuclease domain that recognize a reverse-direction nucleotide sequence spaced 5-6 bp apart from the sequence recognized by the zinc finger proteins. ZFN functions as a dimer in this manner, wherein the zinc finger proteins recognize the target DNA sequence, and the Fokl nuclease domains cleave the intervening region between the binding sites of the zinc finger proteins. When the cleaved site is repaired by HDR, the donor DNA is inserted into the site.

In addition, the components of the gene-editing technology, in the case of TALEN (transcription activator-like effector nuclease) technology, are also composed, similar to ZFN technology, of tandemly arranged DNA-binding domains linked to a Fokl nuclease domain, and (ii) tandemly arranged DNA-binding domains linked to a Fokl nuclease domain that recognize a nucleotide sequence in the opposite orientation, since TALEN also functions as a dimer as in the case of ZFN. Because each DNA-binding domain recognizes only a single nucleotide, in order to provide sequence specificity to the target DNA, the DNA-binding domains of each of the two monomers may comprise at least five, and preferably nine or more, domains. TALEN functions as a dimer, cleaving the region between the binding sites of the two monomers, and donor DNA is inserted into this cleaved site through HDR.

Meanwhile, as a gene-editing technology, RGEN (CRISPR/Cas-derived RNA-guided endonuclease) technology exists in two forms: CRISPR/Cas9 and CRISPR/Cpf1. In the Examples below, gene editing of exons 1, 2, and 8 of the first embryo was carried out using these technologies.

The components of CRISPR/Cas9 technology are Cas9, which has endonuclease activity; crRNA (CRISPR RNA), which recognizes the target DNA sequence; and tracrRNA (trans-activating crRNA), which is partially complementary to crRNA. Since CRISPR/Cas9 technology can also function by using a single guide RNA (sgRNA) formed by combining crRNA and tracrRNA, the components of CRISPR/Cas9 may alternatively consist of Cas9 and sgRNA. Cas9 recognizes PAM (protospacer-associated motif) sequences rich in G, such as NGG, and cleaves the adjacent sequence to create a double-strand break (DSB). This DSB is repaired by HDR, during which donor DNA is inserted at the cleavage site.

CRISPR/Cpf1 technology operates with only Cpf1 and crRNA, and unlike CRISPR/Cas9 technology, does not require tracrRNA. The Cpf1 protein (Cas12a) recognizes PAMs rich in thymidine, such as TTTN (where N represents any nucleotide having the base A, T, G, or C) or TTTV (where V represents any nucleotide having the base A, G, or C), and cleaves sequences distal to the PAM. Unlike Cas9, this cleavage produces cohesive ends with 4-5 nucleotide overhangs, and these ends are repaired by HDR, during which the donor DNA is inserted.

RGEN (CRISPR/Cas-derived RNA-guided endonuclease) technologies such as CRISPR/Cas9 and CRISPR/Cpf1 enable gene editing by simply replacing the RNA component without the need to generate new DNA-binding domain proteins for each target, unlike ZFN or TALEN technologies. For this reason, they are currently widely used in the production of gene therapies and transgenic experimental animals. In the present invention, considering these advantages, it is preferable to use RGEN technology for producing the first embryo, and in fact, in the Examples of the present invention described below, RGEN technology was employed.

In the method of the present invention, when the production of the first embryo is performed by sequentially introducing donor DNAs for the three exons together with the components of the gene-editing technology for each exon, using CRISPR/Cas9 and CRISPR/Cpf1 technologies, such sequential introduction may comprise:
(i) introducing donor DNA for exon 1, together with the components of CRISPR/Cpf1 gene-editing technology for exon 1, namely Cpf1 and crRNA, into an embryo, transplanting it into a surrogate mother to obtain an individual, selecting a founder in which the donor DNA has been inserted, mating the founder with a wild-type mouse to obtain offspring (F1), and mating the offspring with a superovulated female mouse to obtain embryos;
(ii) introducing donor DNA for exon 2, together with the components of CRISPR/Cpf1 gene-editing technology for exon 2, namely Cpf1 and crRNA, into the embryos obtained in step (i), transplanting them into a surrogate mother to obtain individuals, selecting founders in which the donor DNA has been inserted, mating the founders with wild-type mice to obtain offspring (F1), and mating the offspring with superovulated female mice to obtain embryos; and
(iii) introducing donor DNA for exon 8, together with the components of CRISPR/Cas9 gene-editing technology for exon 8, namely Cas9, crRNA, and tracrRNA, into the embryos obtained in step (ii).

In the method of the present invention, the donor DNA for exon 1 may comprise the nucleotide sequence of Sequence ID No. 12, the donor DNA for exon 2 may comprise the nucleotide sequence of Sequence ID No. 13, and the donor DNA for exon 8 may comprise the nucleotide sequence of Sequence ID No. 23.

In the method of the present invention, the crRNA, which is a component of the CRISPR/Cpf1 gene-editing technology for exon 1, may be the crRNA of the nucleotide sequence of Sequence ID No. 8 and/or the crRNA of the nucleotide sequence of Sequence ID No. 9. The crRNA, which is a component of the CRISPR/Cpf1 gene-editing technology for exon 2, may be the crRNA of the nucleotide sequence of Sequence ID No. 10 and/or the nucleotide sequence of Sequence ID No. 11. The crRNA, which is a component of the CRISPR/Cas9 gene-editing technology for exon 8, may be the crRNA of the nucleotide sequence of Sequence ID No. 20 and/or the crRNA of the nucleotide sequence of Sequence ID No. 21, and the tracrRNA, which is a component of the CRISPR/Cas9 gene-editing technology for exon 8, may be the tracrRNA of the nucleotide sequence of Sequence ID No. 22.

The gene-editing components may be introduced into the embryo in the form of plasmids, cosmids, or other expression vectors, or as viral genomes in which the genes are inserted under the control of suitable regulatory sequences such as promoters, thereby being expressed in the form of expression vectors or viral particles. Alternatively, the components may be introduced into the embryo in the form of mRNA expressing the gene-editing components, or directly in the form of proteins, RNA, or the like. When proteins, RNA, or the like are directly introduced, they can act immediately without the need for translation or transcription, and since the genome of the embryo is exposed to the gene-editing components only for a short period of time, off-target editing may be reduced.

For introduction of the gene-editing components into the embryo, a variety of methods known in the art may be used, including virus-mediated transient transfection, microinjection, cell fusion, calcium phosphate precipitation, liposome-mediated transfection, DEAE-dextran-mediated transfection, polybrene-mediated transfection, electroporation, and gene gun.

With respect to gene-editing technologies, a substantial body of literature has been accumulated, including [J Genet Eng Biotechnol. 2021 Dec; 19:125], [Methods Mol Biol. 2022; 2495:29-46], [Int J Mol Sci. 2020 Aug. 7; 21(16):5665], and [Trends Biotechnol. 2013 July; 31(7):397-405], and further details regarding the application of gene-editing technologies may be found therein.

### Advantageous Effects

As described above, according to the present invention, it is possible to provide a transgenic mouse expressing humanized ACE2 that is susceptible to SARS-CoV-2 viral infection, as well as a method for producing the same. The transgenic mouse expressing humanized ACE2 of the present invention reflects symptom severity and mortality patterns according to age, sex, and viral inoculum dose, as observed in human COVID-19 cases, and reproduces the immunopathological characteristics of pulmonary SARS-CoV-2 infection without viral replication or lesions in the brain. Therefore, it can be advantageously used for understanding the pathogenic mechanisms of SARS-CoV-2 and for developing vaccines and therapeutics.

### BRIEF DESCRIPTION OF THE DRAWINGS

FIG. 1 shows differences between the human ACE2 amino acid sequence and the murine ACE2 amino acid sequence.

FIG. 2 shows the results of genotyping analysis of ACE2-humanized mice.

FIG. 3 shows the nucleotide sequences and amino acid sequences of exon 1, exon 2, and exon 8 in wild-type mice, heterozygous ACE2-humanized mice (hACE2(+/−)), and homozygous ACE2-humanized mice (hACE2(+/+)).

FIG. 4 shows the nucleotide sequences and amino acid sequences of wild-type mice and ACE2-humanized mice.

FIG. 5 shows the quantitative results of ACE2 mRNA expression in 10 organs, including the lung, and blood pressure measurements in wild-type mice, heterozygous ACE2-humanized mice (hACE2(+/−)), and homozygous ACE2-humanized mice (hACE2(+/+)).

FIG. 6 shows body weight, mortality, and pathological symptoms in ACE2-humanized mice infected with mouse-adapted SARS-CoV-2 virus.

FIG. 7 shows the presence or absence of encephalitis and the quantitative results of viral loads in organs such as the lung and brain when ACE2-humanized mice were infected with mouse-adapted SARS-CoV-2 virus.

### MODE FOR CARRYING OUT THE INVENTION

The present invention will now be described with reference to the following Examples. However, the present invention is not limited to these Examples.

### <Example> Production of Amino Acid-Substituted ACE2 Humanized Mice

### 1. Experimental Methods

### 1.1 Determination of Amino Acid Substitution Sites in Murine ACE2

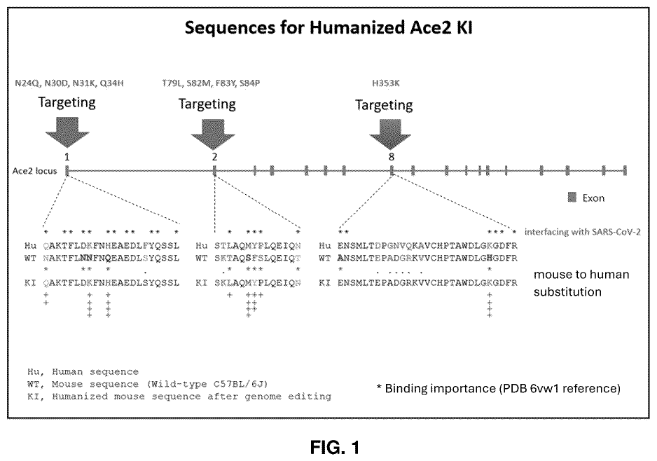

First, as shown in FIG. 1, in order to produce an ACE2 amino acid substitution humanized mouse (Knock-in Mouse, KI), the differences between the human ACE2 amino acid sequence (Sequence ID No. 1) and the murine ACE2 amino acid sequence (Sequence ID No. 2) were identified.

In addition, as shown in FIG. 1, reference was made to the disclosures of [“Structure of the SARS-CoV-2 spike receptor-binding domain bound to the ACE2 receptor” (Nature 581, 215-220 (2020))] and [“Structural basis for the recognition of SARS-CoV-2 by full-length human ACE2” (Science 367,1444-1448 (2020))] to determine amino acid positions involved in binding with the SARS-CoV-2 RBD (SARS-CoV-2 Receptor Binding Domain) (* marks in the figure). Furthermore, structural analyses of the binding between SARS-CoV-2 RBD and human ACE2 protein were conducted independently, and the binding importance of each amino acid position was assessed (+marks in the figure). Binding importance was indicated based on the PDB 6VW1 structure, wherein PDB 6VW1 refers to the registration number of the structure representing the interaction between SARS-CoV-2 RBD and human ACE2 protein in the RCSB Protein Data Bank (RCSB PDB).

Considering the amino acid positions involved in binding, and their binding importance, the murine ACE2 amino acid sequence was edited using the gene-editing technologies described below, such that in exon 1, N24 was substituted with Q, N30 with D, N31 with K, and Q34 with H; in exon 2, T79 was substituted with L, S82 with M, F83 with Y, and S84 with P; and in exon 8, H353 was substituted with K (mouse-to-human substitutions indicated with *). Here, each number refers to the position of the amino acid in the full-length murine ACE2 amino acid sequence (Sequence ID No. 2), the letter to the left of the number indicates the amino acid in the wild-type mouse prior to substitution, and the letter to the right of the number indicates the amino acid in the humanized mouse after substitution. The full-length amino acid sequence of the substituted humanized ACE2 is disclosed in Sequence ID No. 3.

### 1.2 Preparation of Cas Enzymes, crRNAs, and Other Components for Gene-Editing Technology

For the production of amino acid-substituted ACE2 humanized mice, CRISPR-Cpf1 technology and CRISPR-Cas9 technology were employed. CRISPR-Cpf1 technology was used for gene editing of exons 1 and 2 of the ACE2 gene, and CRISPR-Cas9 technology was used for gene editing of exon 8 of the ACE2 gene.

For the application of CRISPR-Cpf1 technology, Cas12a derived from Acidaminococcus sp. BV3L6 (Integrated DNA Technologies, Alt-R® CRISPR-Cas12a (Cpf1), USA) was purchased and used. The target sequences recognized by crRNA are as follows, and the crRNA sequences as well as the donor DNA sequences were synthesized by Integrated DNA Technologies (USA) according to the following sequences.

### Target Sequences for Exon 1 (Sequence ID Nos. 4 and 5, Respectively):

Ace2 E1 Cpf #1:
aacaactttaatcaggaagct (+)
Ace2 E1 Cpf #2:
atcaggaagctgaagacctgt (+)

### Target Sequences for Exon 2 (Sequence ID Nos. 6 and 7, Respectively):

Ace2 E2 Cpf #1:
ttgtagtgagaaactttgggc (−)
Ace2 E2 Cpf #2:
ggcagtcttagactgttcttc (−)

In the above target sequences, (+) denotes the sense strand in the full-length murine genomic sequence, while (−) denotes the antisense strand in the full-length murine genomic sequence.

Using one or more of the above target sequences was intended to increase gene-editing efficiency. That is, CRISPR-Cpf1 technology is more likely to function if at least one of these target sequences is recognized, and the use of overlapping crRNAs improves the efficiency of donor DNA insertion (Exp Mol Med. 2018; 50(4):1-9).

crRNA Sequences for Exon 1 (Sequence ID Nos. 8 and 9, Respectively):

Ace2 E1 Cpf #1:
UAA UUU CUA CUC UUG UAG AUA ACA ACU UUA AUC
AGG AAG CU
Ace2 E1 Cpf #2:
UAA UUU CUA CUC UUG UAG AUA UCA GGA AGC UGA
AGA CCU GU

crRNA Sequences for Exon 2 (Sequence ID Nos. 10 and 11, Respectively):

Ace2 E2 Cpf #1:
UAA UUU CUA CUC UUG UAG AUU UGU AGU GAG AAA
CUU UGG GC
Ace2 E2 Cpf #2:
UAA UUU CUA CUC UUG UAG AUG GCA GUC UUA GAC
UGU UCU UC

### Donor DNA Sequence for Exon 1 (Sequence ID No. 12):

5′-a*t*gtccagctcctcctggctccttctcagccttgttgctgttact
actgctcagtccctcaccgaagaacaggctaagacatttttggacaagtt
caaccacgaagctgaagacctgtcttatcagagttcacttgcttcttgga
atataatactaacattactgaagaaaat*g*c-3′

### Donor DNA Sequence for Exon 2 (Sequence ID No. 13):

5′-t*t*atttacttatttttcagagtgaggctgcagccaaat
ggtctgccttttatgaagaacagtctaagcttgcccagatgta
tccactacaagaaattcaaaacccgatcatcaagcgtcaacta
caggccttcagcaaagtgggtcttcagcactctcagcaga
c*a*a-3′

In the above donor DNAs, “*” indicates a phosphorothioate bond (internal chemical modification). Such chemical modifications are known to improve the efficiency of homology-directed repair (HDR) (Cell Reports. 2016; 14:2263-2272) and were therefore introduced.

The donor DNA for exon 1 replaces, through homology-directed repair (HDR), nucleotides 66 to 126 of the nucleotide sequence of exon 1 (Sequence ID No. 14). The actual substitutions are as follows: nucleotide 66, G→A; nucleotide 70, A→C; nucleotide 72, T→G; nucleotide 75, C→T; nucleotide 87, A→G; nucleotide 88, A→G; nucleotide 93, C→G; nucleotide 96, T→C; nucleotide 99, T→C; nucleotide 102, G→C; and nucleotide 126, A→G. The complete sequence of exon 1 after substitution with the donor DNA is shown in Sequence ID No. 16.

The donor DNA for exon 2 replaces, through HDR, nucleotides 49 to 64 of the nucleotide sequence of exon 2 (Sequence ID No. 15). The actual substitutions are as follows: nucleotide 49, A→C; nucleotide 50, C→T; nucleotide 57, A→G; nucleotide 59, G→T; nucleotide 60, T→G; nucleotide 62, T→A; nucleotide 63, C→T; and nucleotide 64, T→C. The complete sequence of exon 2 after substitution with the donor DNA is shown in Sequence ID No. 17.

For the application of CRISPR-Cas9 technology, Cas9 derived from Streptococcus pyogenes (Integrated DNA Technologies, Alt-R® CRISPR-Cas9, USA) was used. The target sequences recognized by crRNA, as well as the crRNA sequences, tracrRNA sequence, and donor DNA sequence, are as follows. The crRNAs, tracrRNA, and donor DNA were synthesized by Integrated DNA Technologies (USA).

### Target Sequences for Exon 8 (Sequence ID Nos. 18 and 19, Respectively):

Ace2 E8 Cas #1: atctgggacacggagacttc (+)
Ace2 E8 Cas #2: acacggagacttcaggtagt (+)

crRNA and tracrRNA Sequences for Exon 8 (Sequence ID Nos. 20, 21, and 22, Respectively):

Ace2 E8 Cas #1 crRNA:
AUC UGG GAC ACG GAG ACU UCG UUU UAG AGC UAU GCU
Ace2 E8 Cas #2 crRNA:
ACA CGG AGA CUU CAG GUA GUG UUU UAG AGC UAU GCU
tracrRNA:
AGC AUA GCA AGU UAA AAU AAG GCT AGU CCG UUA UCA
ACU UGA AAA AGU GGC ACC GAG UCG GUG CUU U

### Donor DNA Sequence for Exon 8 (Sequence ID No. 23):

5′-c*t*gttggccttcctcatatgactcaaggattctgggaaaactcta
tgctgactgagccagcagatggccggaaagttgtctgccaccccacagct
tgggacctagggaagggcgatttcaggtagttgggttgtcacttacagtg
cagctactaagtgagaaacagtgtatgaagggttcatta*t*a-3′

In the above donor DNA, “*” denotes a phosphorothioate bond (internal chemical modification).

In the above target sequences, (+) denotes the sense strand, and in the donor DNA, “*” denotes a phosphorothioate bond.

The donor DNA for exon 8 replaces, through homology-directed repair (HDR), nucleotides 150 to 165 of the nucleotide sequence of exon 8 (Sequence ID No. 24). The actual substitutions are as follows: nucleotide 150, T→C; nucleotide 153, G→A; nucleotide 156, A→G; nucleotide 157, C→A; nucleotide 159, C→G; nucleotide 162, A→C; and nucleotide 165, C→T. The complete sequence of exon 8 after substitution with the donor DNA is shown in Sequence ID No. 25.

These prepared CRISPR Cas enzymes, crRNAs, donor DNAs, and the like were introduced directly into mouse embryos in the form of proteins or nucleic acids, without mediation by plasmids, as described below.

### 1.3 Production of Amino Acid-Substituted ACE2 Humanized Mice

### 1.3.1 Preparation of Mice

For the production of ACE2 humanized mice, the C57BL/6N strain supplied by Orient Bio Inc. (Korea) was used.

Animal experiments were conducted in the microinjection room and in the Specific Pathogen Free (SPF) animal facility of the Experimental Animal Center at Yonsei University. Experimental mice aged 4-20 weeks were maintained under controlled housing conditions using an Individually Ventilated Cage (IVC) rack system (Threeshine, Korea) in isolated areas with a 12/12 light/dark cycle, a temperature of 23±2° C., humidity of 50%±10%, ammonia levels below 20 ppm, and an airflow velocity of 0.2-0.25 m/sec. Mice were provided with sterilized feed (Purina Korea, Korea) and autoclaved sterilized water.

### 1.3.2 Production of Transgenic Mice

Transgenic mice were generated using the standard gene-editing procedures of GEMCRO Inc. (Korea). Fertilized embryos were obtained by mating male C57BL/6 mice with 3-4-week-old superovulated female C57BL/6 mice. ICR female mice (10-16 weeks old, Orient Bio Inc., Korea) were used as surrogate mothers.

First, crRNAs targeting exon 1, donor DNA, and Cas12a (Cpf1) enzyme protein were microinjected into hundreds of fertilized embryos collected from mice. Thereafter, healthy embryos among the microinjected embryos were transplanted and engrafted into the oviducts of surrogate mothers to develop into individuals. From each newborn mouse, genomic DNA was extracted, and PCR screening was performed using the following primers capable of distinguishing gene-edited sequences on DNA. Sequencing analysis was then outsourced to Cosmo Genetech (Korea) to confirm the corrected sequences.

Subsequently, individuals in which exon 1 of the target gene was corrected on the chromosome (Founder, F0) were identified and secured as founders. These Founder mice (F0) were then mated with wild-type (WT) mice to obtain offspring (F1), thereby generating an F1 transgenic mouse strain in which amino acid substitutions in exon 1 had been introduced.

### Primer Sequences for Analysis of Exon 1 (Sequence ID Nos. 26 and 27, Respectively):

E1 F: tggatgggatcttggcgcac
E1 R: aagcaatcaataaccccctgg

The F1 mice with amino acid substitutions in exon 1 were again mated with 3-4-week-old superovulated C57BL/6 female mice, from which fertilized embryos were collected. crRNAs targeting exon 2, donor DNA, and Cas12a (Cpf1) enzyme protein were microinjected into the embryos, which were then transplanted into the uteri of surrogate mothers for implantation. After birth, genomic DNA of the offspring was analyzed using the primer set described below, and Founder mice (F0) with amino acid substitutions in both exons 1 and 2 were obtained. These Founder mice (F0) with amino acid substitutions in exons 1 and 2 were then mated with wild-type (WT) mice to obtain offspring (F1), thereby generating a transgenic mouse strain with amino acid substitutions in both exons 1 and 2.

### Primer Sequences for Analysis of Exon 2 (Sequence ID Nos. 28 and 29, Respectively):

E2 F: aaccttgaccctcagcaaagta
E2 R: tcaccctacttaggcccagaa

The F1 mice with amino acid substitutions in exons 1 and 2 were again mated with superovulated C57BL/6 female mice, from which fertilized embryos were collected. crRNAs targeting exon 8, together with tracrRNA, donor DNA, and Cas9 enzyme protein, were microinjected into the embryos, which were then transplanted into the uteri of surrogate mothers for implantation. After birth, genomic DNA of the offspring was analyzed using the primer set described below, and Founder mice (F0) with amino acid substitutions in exons 1, 2, and 8 were obtained. These Founder mice (F0) with amino acid substitutions in exons 1, 2, and 8 were then mated with wild-type (WT) mice to obtain offspring (F1), thereby generating a transgenic mouse strain with amino acid substitutions in exons 1, 2, and 8, designated as ACE2 humanized mice (hiACE2 KI mice).

### Primer Sequences for Analysis of Exon 8 (Sequence ID Nos. 30 and 31, Respectively):

E8 F: tgtttctgttggccttcctcat
E8 R: actgtggaccatcttcttttcct

### 1.4 Genotyping Analysis of ACE2 Humanized Mice

Genotyping analysis of ACE2 humanized mice was performed using PCR (polymerase chain reaction). The mice analyzed included wild-type mice (+/+), heterozygous ACE2 humanized mice (+/KI), and homozygous ACE2 humanized mice (KI/KI). Here, heterozygous ACE2 humanized mice (+/KI) were obtained by mating F0 or later-generation heterozygous ACE2 humanized mice (+/KI) with wild-type (+/+) mice, while homozygous ACE2 humanized mice (KI/KI) were obtained by mating female heterozygous ACE2 humanized mice (+/KI) of at least the F1 generation with male heterozygous ACE2 humanized mice (+/KI) of at least the F1 generation.

Genomic DNA (gDNA) was first extracted from tail tissue of the mice using a DNA extraction buffer (25 mM NaOH, 2 mM EDTA) and neutralization buffer (40 mM Tris-HCl). PCR was then performed on the extracted gDNA using the AccuPower Pfu PCR PreMix Kit (Bioneer, K-2024, Korea) according to the manufacturer's protocol.

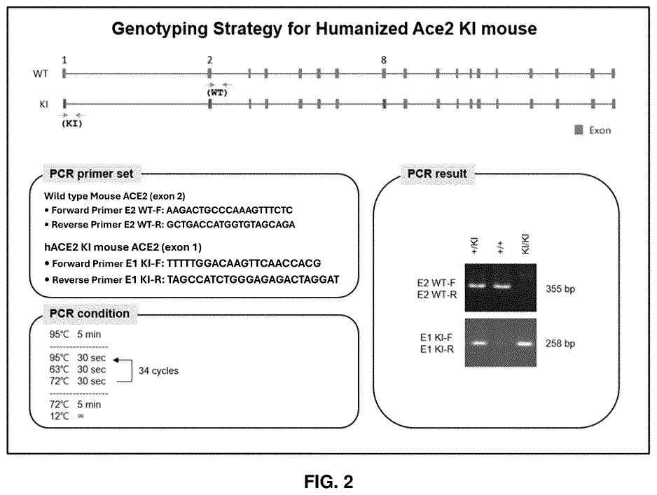

As shown in FIG. 2, genotyping of wild-type (WT) mice was performed using primers targeting the wild-type exon 2 region. Also, as shown in FIG. 2, genotyping of ACE2 humanized (KI) mice was performed using primers targeting the humanized exon 1 region. The target sequence region of wild-type (WT) exon 2 spans from nucleotide 46 of exon 2 to nucleotides located within the intron between exon 2 and exon 3. The forward primer was designed to recognize nucleotides 46-65 of exon 2, which include the point mutation target site introduced by homology-directed repair (HDR), and therefore does not amplify the humanized exon 2 of ACE2 humanized mice. The target sequence region of humanized (KI) exon 1 spans from nucleotide 82 of exon 1 to nucleotides located within the intron between exon 1 and exon 2. The forward primer was designed to recognize nucleotides 82-103 of exon 1, which include the point mutation target site introduced by HDR, and therefore does not amplify exon 1 of wild-type mice.

The primer sequences are shown below, and PCR was performed under the conditions indicated in FIG. 2: initial denaturation at 95° C. for 5 minutes (1 cycle), followed by 34 cycles of 95° C. for 30 seconds (denaturation), 63° C. for 30 seconds (annealing), 72° C. for 30 seconds (extension), and a final extension at 72° C. for 5 minutes (1 cycle).

### Primer Sequences for Wild-Type Mouse Exon 2 (Sequence ID Nos. 32 and 33, Respectively):

E2 WT-F: AAG ACT GCC CAA AGT TTC TC
E2 WT-R: GCT GAC CAT GGT GTA GCA GA

Primer Sequences for Humanized Mouse (hACE2 KI) Exon 1 (Sequence ID Nos. 34 and 35, Respectively):

E1 KI-F: TTT TTG GAC AAG TTC AAC CAC G
E1 KI-R: TAG CCA TCT GGG AGA GAC TAG GAT

The PCR products were subjected to agarose gel electrophoresis after the addition of Safe Shine Green (BioSesang, Korea) at 10,000× concentration, and the DNA bands were visualized to confirm amplification.

The PCR product targeting the humanized exon 1 (E1) of ACE2 humanized mice had a size of 258 bp, while the PCR product targeting the wild-type exon 2 (E2) of wild-type mice had a size of 355 bp.

As shown in FIG. 2, analysis of the PCR products demonstrated that wild-type mice (+/+) yielded products only in the PCR reaction targeting wild-type exon 2. Heterozygous mice (+/KI) yielded products in both PCR reactions targeting humanized exon 1 and wild-type exon 2. Homozygous ACE2 humanized mice (KI/KI) yielded products only in the PCR reaction targeting humanized exon 1.

Genotyping analysis of ACE2 humanized mice was further confirmed by Sanger sequencing (Proc Natl Acad Sci USA. 1977 Dec; 74(12): 5463-5467).

For sequencing, the extracted gDNA described above was used. PCR reactions were carried out using the primers listed below for exons 1, 2, and 8, respectively, to generate amplicons for sequencing. The PCR conditions for amplification were: initial denaturation at 95° C. for 5 minutes (1 cycle), followed by 30 cycles of denaturation at 95° C. for 30 seconds, annealing at 60° C. for 30 seconds, and extension at 72° C. for 40 seconds, with a final extension at 72° C. for 5 minutes (1 cycle).

### Primer Sequences for Sequencing Exon 1 (Sequence ID Nos. 36 and 37, Respectively):

E1 F: GTCCAGCAGCTTGTTTACTGTTC
E1 R: GGCAGTCACTCATCCTCACA

### Primer Sequences for Sequencing Exon 2 (Sequence ID Nos. 38 and 39, Respectively):

E2 F: TGACCCTCAGCAAAGTAAAGG
E2 R: ACCCAAGTGTCTTCTTTCAACC

Primer Sequences for Sequencing Exon 8 (Sequence ID Nos. 40 and 41, Respectively):

E8 F: CGTGCAAAAGCCTACTTCTCT
E8 R: CAAACCTTTCCTAACAGGGGG

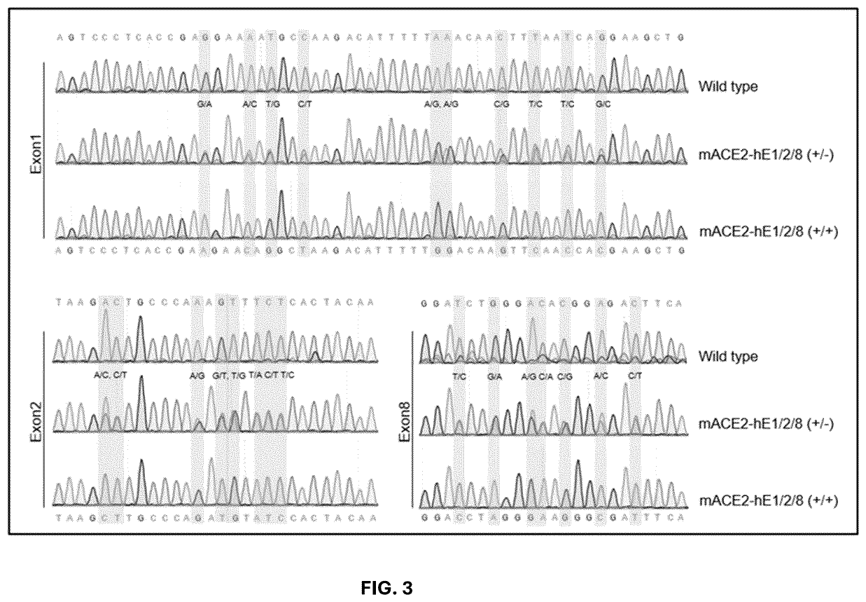

Sanger sequencing was then performed on these PCR amplicons, and the results are shown in FIG. 3.

As confirmed in FIG. 3, in the gDNA of wild-type mice, the nucleotides at positions 66, 70, 72, 75, 87, 88, 93, 96, 99, 102, and 126 of exon 1 were identified as G, A, T, C, A, A, C, T, T, G, and A, respectively; the nucleotides at positions 49, 50, 57, 59, 60, 62, 63, and 64 of exon 2 were identified as A, C, A, G, T, T, C, and T, respectively; and the nucleotides at positions 150, 153, 156, 157, 159, 162, and 165 of exon 8 were identified as T, G, A, C, C, A, and C, respectively.

In the gDNA of homozygous (hACE2 (KI/KI)) ACE2 humanized mice, the nucleotides at positions 66, 70, 72, 75, 87, 88, 93, 96, 99, 102, and 126 of exon 1 were converted as follows: G→A, A→C, T→G, C→T, A→G, A→G, C→G, T→C, T→C, G→C, and A→G, respectively. In exon 2, the nucleotides at positions 49, 50, 57, 59, 60, 62, 63, and 64 were converted as follows: A→C, C→T, A→G, G→T, T→G, T→A, C→T, and T→C, respectively. In exon 8, the nucleotides at positions 150, 153, 156, 157, 159, 162, and 165 were converted as follows: T→C, G→A, A→G, C→A, C→G, A→C, and C→T, respectively. In the above, the nucleotide to the left of the arrow represents the wild-type nucleotide, and the nucleotide to the right of the arrow represents the humanized mouse nucleotide.

In the gDNA of heterozygous (hACE2 (+/KI)) ACE2 humanized mice, the nucleotides at positions 66, 70, 72, 75, 87, 88, 93, 96, 99, 102, and 126 of exon 1 were confirmed as G/A, A/C, T/G, C/T, A/G, A/G, C/G, T/C, T/C, G/C, and A/G, respectively. In exon 2, the nucleotides at positions 49, 50, 57, 59, 60, 62, 63, and 64 were confirmed as A/C, C/T, A/G, G/T, T/G, T/A, C/T, and T/C, respectively. In exon 8, the nucleotides at positions 150, 153, 156, 157, 159, 162, and 165 were confirmed as T/C, G/A, A/G, C/A, C/G, A/C, and C/T, respectively. In the above, the nucleotide to the left of the slash (/) represents the wild-type nucleotide, and the nucleotide to the right of the slash (/) represents the humanized mouse nucleotide.

### 1.5 Relative Quantification of ACE2 mRNA in Mouse Organs

Eleven-week-old female wild-type mice, heterozygous ACE2 humanized mice, and homozygous ACE2 humanized mice were sacrificed, respectively.

From each mouse, a portion or the entirety of ten organs—lung, airway, heart, kidney, liver, spleen, small intestine, large intestine, brain, and stomach—was homogenized, and total RNA was extracted from the homogenized tissue using TRIzol reagent (Thermo Scientific, USA).

Between 50 ng and 1 μg of RNA from the extracted organs was used for reverse transcription PCR (RT-PCR) to obtain cDNA. Real-time PCR targeting mRNA of humanized ACE2 (hACE2), murine ACE2 (mACE2), and beta-actin was then performed using the obtained cDNA and the primers disclosed below, together with SYBR Green dye (Thermo Scientific, USA).

Real-Time PCR Primer Sequences for Humanized ACE2 (hACE2) (Sequence ID Nos. 42 and 43, Respectively):

hACE2 F: TTTTTGGACAAGTTCAACCACG
hACE2 R: CTGGATTTCTTGTAGTGGATACATC

Real-Time PCR Primer Sequences for Murine ACE2 (mACE2) (Sequence ID Nos. 44 and 45, Respectively):

mACE2 F: CCAAGACATTTTTAAACAACTTTAATCAG
mACE2 R: GTCTGGATTTCTTGTAGTGAGA

### Real-Time PCR Primer Sequences for Beta-Actin (Sequence ID Nos. 46 and 47, Respectively):

ACTB F: TGTTACCAACTGGGACGACATG
ACTB R: GGGGTGTTGAAGGTCTCAAAC

Specifically, the real-time polymerase chain reaction (PCR) conditions were as follows:

For hACE2: (1) denaturation at 95° C. for 10 minutes; (2) denaturation at 95° C. for 15 seconds; (3) annealing and extension at 60° C. for 1 minute; (4) plate reading; and (5) repetition of steps (2)-(4) for 39 cycles.

For mACE2: (1) denaturation at 95° C. for 10 minutes; (2) denaturation at 95° C. for 15 seconds; (3) annealing and extension at 55° C. for 1 minute; (4) plate reading; and (5) repetition of steps (2)-(4) for 39 cycles.

For beta-actin: (1) denaturation at 95° C. for 10 minutes; (2) denaturation at 95° C. for 15 seconds; (3) annealing and extension at 61° C. for 1 minute; (4) plate reading; and (5) repetition of steps (2)-(4) for 39 cycles.

After completion of all reactions under the above real-time PCR conditions, a melting curve was generated by plate reading at every 0.5° C. increment from 65° C. to 95° C. to confirm the absence of nonspecific amplification.

The forward primer for hACE2 was designed to target nucleotides 82-103 within exon 1 (Sequence ID No. 42), and the reverse primer was designed to target nucleotides 76-100 within exon 2 (Sequence ID No. 43). The forward primer for mACE2 was designed to target nucleotides 74-102 within exon 1 (Sequence ID No. 44), and the reverse primer was designed to target nucleotides 62-83 within exon 2 (Sequence ID No. 45). In this way, the primers for hACE2 specifically amplified only hACE2, and the primers for mACE2 specifically amplified only mACE2.

The relative amounts of hACE2 mRNA and mACE2 mRNA were normalized to the amount of beta-actin mRNA in each sample, and the values were further normalized by dividing by the lowest relative mRNA amount among all samples.

### 1.6 Measurement of Blood Pressure in Mice

Wild-type mice aged 2-3 months and 5-7 months, as well as amino acid-substituted homozygous ACE2 humanized mice, were used for the measurements.

Each mouse was placed in a restrainer (Kent Scientific, USA) and acclimated. A hot pack or heating device was then used to warm the tail to 30° C.

For consistent blood pressure measurements, the mice were stabilized prior to measurement. A blood pressure cuff (Kent Scientific, USA) (X-small occlusion cuff, up to 25 g) was attached to the tail of each mouse. Blood pressure was measured using the CODA non-invasive blood pressure system (Kent Scientific, USA) and CODA2.2 software (Kent Scientific, USA). The measurement procedure was repeated at least 10 times, and only the stable readings were averaged and used as results. The final blood pressure values were obtained as the mean of at least seven repeated measurements.

### 1.7 Mouse Infection Experiments

All infection experiments were conducted using homozygous ACE2 humanized mice.

Female and male ACE2 humanized mice aged 2-3 months and 5-7 months were anesthetized by intraperitoneal injection of a ketamine/xylazine mixture. Mouse-adapted SARS-CoV-2 virus was diluted in PBS at concentrations of 1000 FFU/50 μl, 10,000 FFU/50 μl, and 100,000 FFU/50 μl. The mouse-adapted SARS-CoV-2 was generated in-house by intranasal infection of mice, followed by homogenization of infected lung tissue 2 days post-infection, and reinfection of new mice with the homogenates. This process was repeated 30 consecutive times to establish adaptation.

The anesthetized mice were intranasally infected with 50 μl of the virus suspension at each concentration. For a subset of the infected mice, body weight and survival were monitored once daily for 13 days. Another subset of mice was sacrificed on day 3 post-infection, and the lungs were collected.

### 1.8 Histopathological Analysis of Mouse Lungs

A portion of the lungs from the infected mice was fixed in 1 ml of 4% paraformaldehyde (PFA) at room temperature for at least one day. The fixed lungs were dehydrated and paraffin-embedded to prepare paraffin blocks. These blocks were sectioned at a thickness of 4 μm, mounted on slides, and subjected to hematoxylin and eosin (H&E) staining. The H&E-stained slides were submitted to a professional pathologist (Department of Pathology, Seoul National University College of Medicine) for inflammation analysis.

### 1.9 Quantification of SARS-CoV-2 RNA in Mouse Lung, Brain, and Other Tissues

Portions of lung and brain tissue from the infected mice were homogenized, immersed in 1 ml of TRIzol reagent (Thermo Scientific, USA), and total RNA was extracted according to the manufacturer's instructions. Approximately 1 μg of RNA was used for reverse transcription PCR to obtain cDNA.

The obtained cDNA was subjected to real-time PCR targeting the SARS-CoV-2 RdRp region using the following primers and probe: forward primer (5′-gtgaaatggtcatgtgtggcgg-3′, Sequence ID No. 48), reverse primer (5′-caaatgttaaaaacactattagcata-3′, Sequence ID No. 49), and probe (5′-FAM-caggtggaacctcatcaggagatgc-BHQ1-3′, Sequence ID No. 50). The PCR reaction conditions were: (1) denaturation at 95° C. for 10 minutes, (2) denaturation at 95° C. for 15 seconds, (3) annealing and extension at 55° C. for 1 minute, (4) plate reading, and (5) repetition of steps (2)-(4) for 39 cycles.

Absolute quantification of viral RNA was performed using a standard DNA with a known copy number, which was included in the experiment to generate a standard curve.

### 2. Experimental Results

### 2.1 Amino Acid Sequences of Exons 1, 2, and 8 of ACE2 in Humanized Mice and the Corresponding Gene Sequences

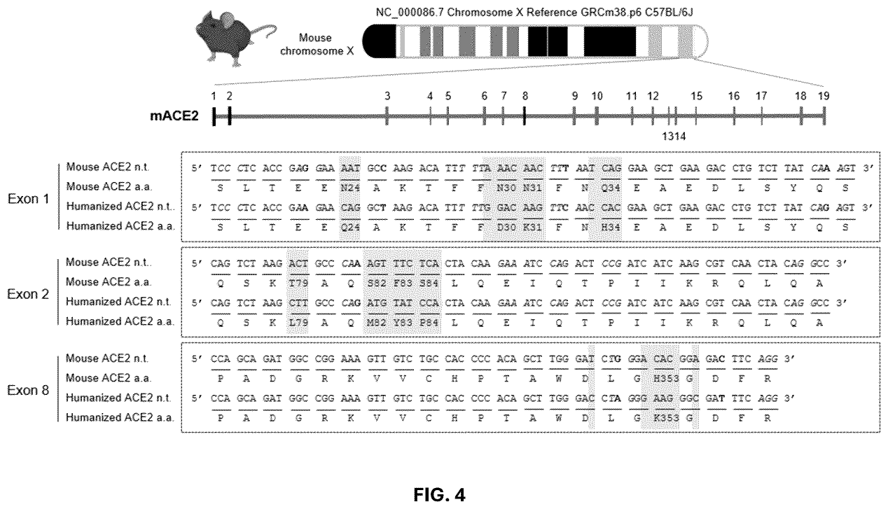

As shown in FIG. 4, ACE2 humanized mice were generated by substituting a total of nine amino acid residues within mouse ACE2 using genome editing technology, specifically N24Q, N30D, N31 K, and Q34H in exon 1; T79L, S82M, F83Y, and S84P in exon 2; and H353K in exon 8.

In FIG. 4, the boxed regions within the gene sequence indicate the loci genetically engineered to replace the mouse ACE2 sequence with the human ACE2 sequence. The bold nucleotides represent synonymous mutations introduced to prevent re-cutting by the CRISPR system and to facilitate genotyping. The italicized nucleotides indicate PAM sequences for Cas9 or Cpf1.

Accordingly, the humanized mice of the present invention possess an ACE2 amino acid sequence in which exon 1 is substituted with N24Q, N30D, N31 K, and Q34H; exon 2 with T79L, S82M, F83Y, and S84P; and exon 8 with H353K. The ACE2 gene sequence is characterized by the following nucleotide substitutions: in exon 1, nucleotides at positions 66, 70, 72, 75, 87, 88, 93, 96, 99, 102, and 126 were converted from G→A, A→C, T→G, C→T, A→G, A→G, C→G, T→C, T→C, G→C, and A→G, respectively; in exon 2, nucleotides at positions 49, 50, 57, 59, 60, 62, 63, and 64 were converted from A→C, C→T, A→G, G→T, T→G, T→A, C→T, and T→C, respectively; and in exon 8, nucleotides at positions 150, 153, 156, 157, 159, 162, and 165 were converted from T→C, G→A, A→G, C→A, C→G, A→C, and C→T, respectively.

### 2.2 Relative Quantification of ACE2 mRNA in Mouse Organs

The expression levels of ACE2 in 10 different organs of ACE2 humanized mice were compared to those of wild-type mice at the mRNA level using real-time PCR. The 10 organs included the lung, airway, heart, kidney, liver, spleen, small intestine, large intestine, brain, and stomach.

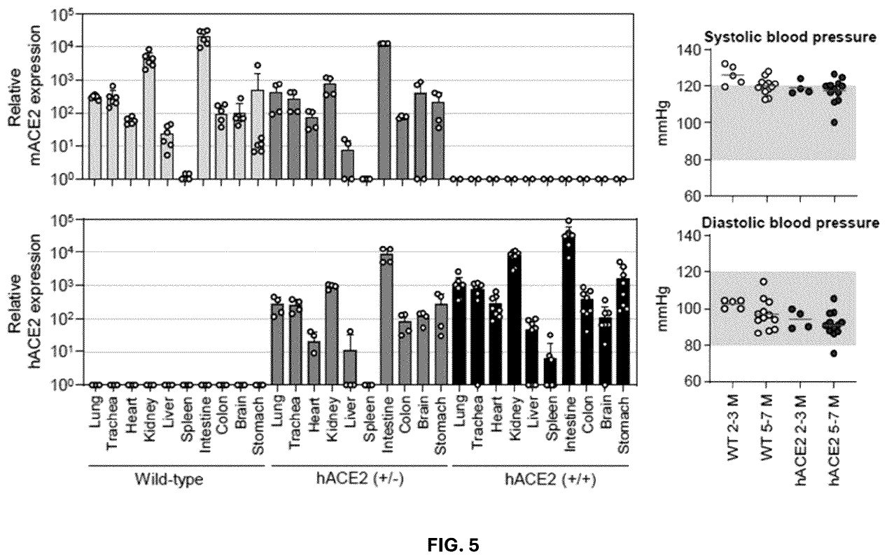

As shown in FIG. 5, the expression levels of humanized ACE2 in these 10 organs were similar across wild-type mice, heterozygous ACE2 humanized mice, and homozygous ACE2 humanized mice. This indicates that no artificial alteration in ACE2 expression was induced during the generation of ACE2 humanized mice.

### 2.3 Blood Pressure Measurement in Mice

As shown in FIG. 5, blood pressure measurements in wild-type mice and ACE2 humanized mice demonstrated that all mice, regardless of age, maintained normal ranges of systolic blood pressure (≤120 mmHg) and diastolic blood pressure (≤80 mmHg).

These normal blood pressure values indicate that the intrinsic function of ACE2 is preserved in humanized mice.

### 2.4 Results of Mouse Infection Experiments

When ACE2 humanized mice were infected with mouse-adapted SARS-CoV-2 virus, serially passaged 30 times in mouse lungs, they recapitulated severe COVID-19.

In 5-7-month-old (5-7 M) ACE2 humanized mice, the severity of disease, ranging from mild illness to mortality, reflected human COVID-19 progression, depending on the viral inoculum dose and the sex of the mice.

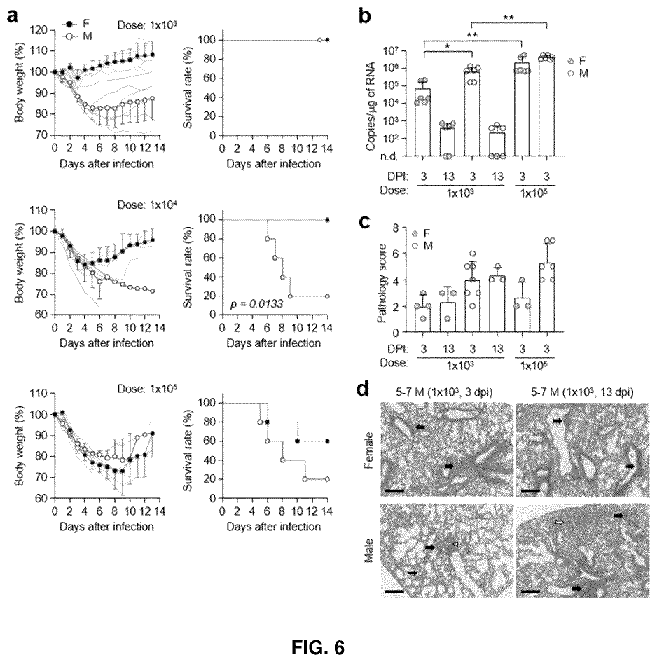

As shown in FIGS. 6a and 6b, infection with 1×10³ FFU did not cause mortality in any mice. Male mice, however, exhibited sustained weight loss and higher viral titers in the lungs at 3 days post infection (DPI) compared to female mice.

As shown in FIGS. 6c and 6d, lung inflammation persisted in male mice up to day 13 post infection. The pathology score in FIG. 6c was derived from four criteria: (1) diffuse alveolar damage including edema, hyaline membranes, and necrosis; (2) bronchitis; (3) monocyte infiltration; and (4) vascular changes such as thrombosis and hemorrhage. Each lesion was graded as follows: 0=no lesion, 1=minimal (<5% of total lung sections), 2=mild (5-33%), 3=moderate (33-66%), and 4=severe (>67%). In FIG. 6d, black arrows indicate monocyte infiltration, white arrows indicate diffuse alveolar damage including edema, hyaline membranes, and necrosis, and arrowheads indicate bronchitis.

As shown in FIG. 6a, infection with 1×10⁴ FFU caused 80% mortality in male mice, whereas female mice exhibited approximately 15% weight loss before recovery.

As shown in FIG. 6a, infection with 1×10⁵ FFU resulted in 40% mortality in female mice and 20% in male mice.

As shown in FIG. 6b, mice infected with 1×10⁵ FFU displayed higher levels of viral RNA in the lungs at 3 DPI compared to those infected with 1×10³ FFU, in both sexes.

As shown in FIG. 6c, comparison of lung pathology scores between 1×10⁵ FFU- and 1×10³ FFU-infected mice revealed that male mice infected with 1×10⁵ FFU exhibited the highest lung pathology scores.

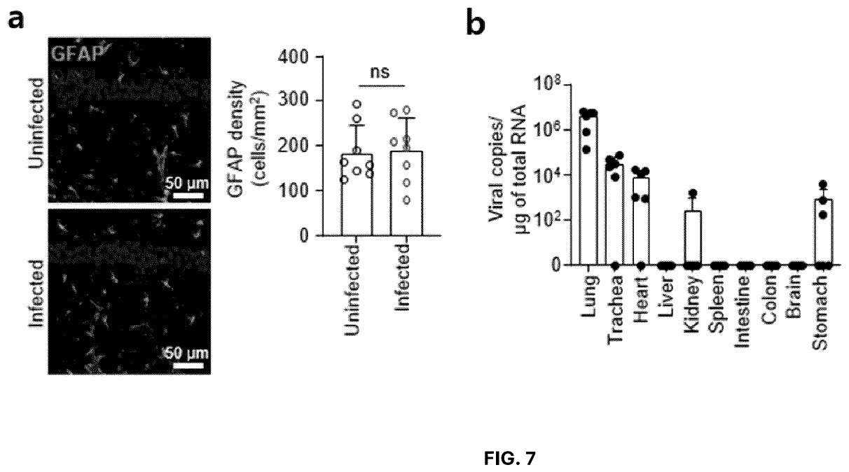

Meanwhile, as shown in FIG. 7, 5-7-month-old ACE2 humanized mice did not develop encephalitis upon induction of severe COVID-19. Specifically, as shown in FIG. 7a, no increase in astrocytic activity (as measured by GFAP density index) was detected in ACE2 humanized mice infected with 1×10⁵ FFU of mouse-adapted virus, indicating that neuroinflammation did not occur. As shown in FIG. 7b, no viral RNA was detected in the brains of ACE2 humanized mice infected with 1×10⁵ FFU of mouse-adapted virus. The absence of brain lesions and viral detection in the brain suggests that this model, unlike conventional severe COVID-19 mouse models, more accurately recapitulates human severe COVID-19, in which pneumonia is the primary cause of morbidity and mortality.

In this specification, the sequence list is as follows.

| Sequence | |
|---|---|
| No. | Description |
| 1 | Human ACE2 sequence |
| 2 | Mouse ACE2 sequence |
| 3 | Humanized ACE2 sequence |
| 4 | Exon 1 target sequence 1 |
| 5 | Exon 1 target sequence 2 |
| 6 | Exon 2 target sequence 1 |
| 7 | Exon 2 target sequence 2 |
| 8 | Exon 1 crRNA sequence 1 |
| 9 | Exon 1 crRNA sequence 2 |
| 10 | Exon 2 crRNA sequence 1 |
| 11 | Exon 2 crRNA sequence 2 |
| 12 | Exon 1 donor DNA sequence |
| 13 | Exon 2 donor DNA sequence |
| 14 | Nucleotide sequence of mouse exon 1 |
| 15 | Nucleotide sequence of mouse exon 2 |
| 16 | Nucleotide sequence of humanized exon 1 |
| 17 | Nucleotide sequence of humanized exon 2 |
| 18 | Exon 8 target sequence 1 |
| 19 | Exon 8 target sequence 2 |
| 20 | Exon 8 crRNA sequence 1 |
| 21 | Exon 8 crRNA sequence 2 |
| 22 | Exon 8 tracrRNA sequence 2 |
| 23 | Exon 8 donor DNA sequence |
| 24 | Nucleotide sequence of mouse exon 8 |
| 25 | Nucleotide sequence of humanized exon 8 |
| 26, 27 | Primer sequences for exon 1 analysis |
| 28, 29 | Primer sequences for exon 2 analysis |
| 30, 31 | Primer sequences for exon 8 analysis |
| 32, 33 | Primer sequences for wild-type mouse exon 2 |
| 34, 35 | Primer sequences for humanized mouse (hACE2 KI) exon 1 |
| 36, 37 | Sequencing primer sequences for exon 1 |
| 38, 39 | Sequencing primer sequences for exon 2 |
| 40, 41 | Sequencing primer sequences for exon 8 |
| 42, 43 | PCR primer sequences for humanized ACE2 |
| 44, 45 | PCR primer sequences for mouse ACE2 |
| 46, 47 | PCR primer sequences for Beta-actin |
| 48, 49 | PCR primer sequences for SARS-CoV-2 RdRp |
| 50 | PCR probe sequence for SARS-CoV-2 RdRp |
| 51 | Gene encoding humanized ACE2 (including introns) |
| 52 | Gene encoding humanized ACE2 (cDNA, excluding introns) |

### INDUSTRIAL APPLICABILITY

The humanized ACE2-expressing transgenic mouse of the present invention reflects symptom severity and mortality patterns according to age, sex, and viral inoculation dose as observed in human COVID-19 cases, and reproduces the immunopathological characteristics of SARS-CoV-2 infection in the lungs without viral replication or lesions in the brain. Therefore, it can be usefully employed for understanding the pathogenic mechanisms of SARS-CoV-2 and for the development of vaccines or therapeutics.

1. A transgenic mouse expressing humanized ACE2 consisting of the amino acid sequence of Sequence ID No. 3, which is susceptible to infection by the SARS-CoV-2 virus.

2. The transgenic mouse of claim 1, wherein the transgenic mouse is derived from and produced by an embryo modified with a gene encoding the humanized ACE2 (Founder, F0).

3. The transgenic mouse of claim 1, wherein the transgenic mouse is produced through one or more generations of inbreeding or outbreeding from a mouse (Founder, F0) derived from an embryo modified with a gene encoding the humanized ACE2, and expresses the humanized ACE2.

4. The transgenic mouse of claim 1, wherein the transgenic mouse comprises the gene encoding the humanized ACE2 in a heterozygous or homozygous form.

5. The transgenic mouse of claim 1, wherein the transgenic mouse comprises the gene encoding the humanized ACE2 in a heterozygous or homozygous form, and wherein the gene encoding the ACE2 comprises the nucleotide sequences of exon 1 of Sequence ID No. 16, exon 2 of Sequence ID No. 17, and exon 8 of Sequence ID No. 25.

6. The transgenic mouse of claim 1, wherein the transgenic mouse comprises the gene encoding the humanized ACE2 in a heterozygous or homozygous form, and wherein the gene encoding the ACE2 comprises the nucleotide sequence of Sequence ID No. 52.

7. A mouse embryo comprising, in a heterozygous or homozygous form, a gene encoding the humanized ACE2 consisting of the amino acid sequence of Sequence ID No. 3.

8. A pluripotent stem cell comprising, in a heterozygous or homozygous form, a gene encoding the humanized ACE2 consisting of the amino acid sequence of Sequence ID No. 3.

9. A method for producing a transgenic mouse, comprising breeding two humanized ACE2-expressing transgenic mice according to claim 1 to obtain offspring.

10. A method for producing a transgenic mouse, comprising breeding a humanized ACE2-expressing transgenic mouse according to claim 1 with a non-transgenic wild-type mouse to obtain offspring.

11. A method for producing a transgenic mouse, comprising preparing a mouse embryo according to claim 7 and obtaining a mature mouse individual from the embryo.

12. A method for producing a transgenic mouse, comprising preparing a pluripotent stem cell according to claim 8 and obtaining a mature mouse individual from the pluripotent stem cell.

13. A method for producing a transgenic mouse, comprising:
(a) introducing into a mouse embryo donor DNAs together with the components of gene-editing technology for each exon, wherein
(i) the donor DNA for exon 1 comprises a nucleotide sequence encoding amino acids 24-34 including the four human amino acids 24Q, 30D, 31 K, and 34H, so that the four mouse amino acids N24, N30, N31, and Q34 are substituted with the human amino acids 24Q, 30D, 31 K, and 34H;
(ii) the donor DNA for exon 2 comprises a nucleotide sequence encoding amino acids 79-84 including the four human amino acids 79L, 82M, 83Y, and 84P, so that the four mouse amino acids T79, S82, F83, and S84 are substituted with the human amino acids 79L, 82M, 83Y, and 84P; and
(iii) the donor DNA for exon 8 comprises a nucleotide sequence encoding the human amino acid 353K, so that the mouse amino acid H353 is substituted with the human amino acid 353K,
wherein the numbers indicate the amino acid positions in the mouse ACE2 amino acid sequence of Sequence ID No. 1 or the humanized ACE2 amino acid sequence of Sequence ID No. 3, the amino acid to the left of the number denotes the wild-type mouse amino acid, and the amino acid to the right of the number denotes the humanized mouse amino acid;
(b) implanting the mouse embryo into a surrogate uterus to obtain individuals; and
(c) selecting individuals (founders) in which the donor DNA has been inserted.

14. The method of claim 13, wherein the donor DNAs for the three exons are introduced into the embryo together with the gene-editing components for each exon either sequentially or simultaneously.

15. The method of claim 13, wherein the gene-editing components comprise Cpf1 and crRNA constituting CRISPR/Cpf1 gene-editing technology.

16. The method of claim 13, wherein the gene-editing components comprise Cas9, crRNA, and tracrRNA constituting CRISPR/Cas9 gene-editing technology.

17. The method of claim 13, wherein step (a) is carried out by sequentially introducing the donor DNAs for the three exons together with the respective gene-editing components into embryos, and the sequential introduction comprises:
(i) introducing the donor DNA for exon 1 together with Cpf1 and crRNA, which are components of CRISPR/Cpf1 gene-editing technology for exon 1, into embryos, implanting the embryos into a surrogate to obtain individuals, selecting founders in which the donor DNA has been inserted, breeding the founders with wild-type mice to produce F1 offspring, and then mating the F1 offspring with superovulated female mice to obtain embryos;
(ii) introducing the donor DNA for exon 2 together with Cpf1 and crRNA, which are components of CRISPR/Cpf1 gene-editing technology for exon 2, into the embryos obtained in step (i), implanting the embryos into a surrogate to obtain individuals, selecting founders in which the donor DNA has been inserted, breeding the founders with wild-type mice to produce F1 offspring, and then mating the F1 offspring with superovulated female mice to obtain embryos; and
(iii) introducing the donor DNA for exon 8 together with Cas9, crRNA, and tracrRNA, which are components of CRISPR/Cas9 gene-editing technology for exon 8, into the embryos obtained in step (ii).

18. The method of claim 13, wherein the donor DNA for exon 1 comprises the nucleotide sequence of Sequence ID No. 12, the donor DNA for exon 2 comprises the nucleotide sequence of Sequence ID No. 13, and the donor DNA for exon 8 comprises the nucleotide sequence of Sequence ID No. 23.

19. The method of claim 17, wherein:
the crRNAs, as components of CRISPR/Cpf1 gene-editing technology for exon 1, are two crRNAs having the nucleotide sequences of Sequence ID Nos. 8 and 9;
the crRNAs, as components of CRISPR/Cpf1 gene-editing technology for exon 2, are two crRNAs having the nucleotide sequences of Sequence ID Nos. 10 and 11;
the crRNAs, as components of CRISPR/Cas9 gene-editing technology for exon 8, are two crRNAs having the nucleotide sequences of Sequence ID Nos. 20 and 21; and
the tracrRNA, as a component of CRISPR/Cas9 gene-editing technology for exon 8, is a tracrRNA having the nucleotide sequence of Sequence ID No. 22.

20. A cell isolated and obtained from the humanized ACE2-expressing transgenic mouse according to claim 1.
